(19) United States
(12) Patent Application Publication
(10) Pub. No.: US 2021/0401937 A1
(43) Pub. Date: Dec. 30, 2021

(54) COMPOSITION AND METHOD FOR ARTHRITIS

(71) Applicant: The Regents of the University of California, Oakland, CA (US)

(72) Inventors: B. Chia SOO, Los Angeles, CA (US); Kang TING, Los Angeles, CA (US); Zhong ZHENG, Van Nuys, CA (US); Chenshuang LI, Van Nuys, CA (US)

(73) Assignee: The Regents of the University of California, Oakland, CA (US)

(21) Appl. No.: 17/289,664

(22) PCT Filed: Oct. 31, 2019
(86) PCT No.: PCT/US2019/059108

§ 371 Date: Apr. 28, 2021

Related U.S. Application Data
(60) Provisional application No. 62/753,158, filed on Oct. 31, 2018.

(51) Int. Cl. A61K 38/18; A61K 45/06; A61P 19/02

(52) U.S. Cl. CPC A61K 38/1875; A61P 19/02; A61K 45/06

(57) ABSTRACT

Embodiments of the present invention provide a method treating cartilage arthritis in a subject in need thereof, which method comprising administering an effective amount of NELL-1 protein or peptide to the subject to ameliorate or treat at least an osteoinflammatory cartilage condition in the subject. The present invention also provides a composition and method therefor.

### GOVERNMENT SUPPORT CLAUSE

This invention was made with government support under Grant Numbers AR061399, AR066782, and AR068835, awarded by the National Institutes of Health. The government has certain rights in the invention.

### FIELD OF THE INVENTION

The invention relates generally to biomedical sciences and technologies and particularly to tissue harvesting and tissue graft application devices and methods.

### BACKGROUND OF THE INVENTION

Arthritis appears in over 100 identified diseases that can damage any joint in the body, causing inflammation that results in pain, stiff-ness, swelling, and decreased motion [1,2]. As the leading cause of disability among adults, arthritis has been diagnosed in more than 10 million people in the United Kingdom [1] and approximately 54.4 million people in the United States [2,3]. In particular, osteoarthritis (OA) is the most common form of arthritis and affects around 18% of women and 10% of men over the age of 60 [4]. Unfortunately, the traditional use of analgesia is insufficient for curative treatment since it does not reduce inflammation and cartilage damage [5-7]. Multiple adverse side-effects in the musculoskeletal, cardiovascular, and gastrointestinal systems [8-10] challenge the use of glucocorticoids as safe arthritis treatments, and non-steroidal anti-inflammatory drugs (NSAIDs) reduce pain and inflammation in the short-term but do not effectively control arthritis progression [6]. Even more disappointing, the efficacy of disease-modifying antirheumatic drugs (DMARDs) that postpone rheumatoid arthritis (RA) progression by slowing or suppressing inflammation has not been replicated in OA clinical trials via systemic or local administration [11-13]. This likely occurs because these therapeutics do not directly manage cartilage destruction—the primary cause of OA [4,6,7,14].

Therefore, the need for additional method and compositions for pro-chondrogenic and anti-inflammatory agents is manifest.

The embodiments described below address the above-identified concerns and needs.

### SUMMARY OF THE INVENTION

In one aspect of the present invention, it is provided a method of treating a cartilage arthritis condition in a subject in need thereof, comprising:

administering an effective amount of a pro-chondrogenic agent to the subject; and

administering an effective amount of an osteoinflammation preventive agent to the subject, thereby ameliorating or treating the cartilage arthritis condition in the subject, wherein the pro-chondrogenic agent promotes cartilage regeneration in the subject, and wherein the osteoinflammation preventive agent reduces or decreases the cartilage arthritis condition in the subject.

In some embodiments of the invention method, optionally in combination with any of the various embodiments disclosed herein, the osteoinflammation preventive agent comprises NELL-1 protein or peptide (SEQ ID NO: 1).

In some embodiments of the invention method, optionally in combination with any of the various embodiments disclosed herein, the pro-chondrogenic agent comprises NELL-1 protein or peptide.

In some embodiments of the invention method, optionally in combination with any of the various embodiments disclosed herein, the NELL-1 is in a formulation for injection at a site of cartilage arthritis of the subject.

In some embodiments of the invention method, optionally in combination with any of the various embodiments disclosed herein, the NELL-1 is administered to the subject by injection at least one time per day over a treatment course.

In some embodiments of the invention method, optionally in combination with any of the various embodiments disclosed herein, the cartilage arthritis is osteoarthritis (OA).

In some embodiments of the invention method, optionally in combination with any of the various embodiments disclosed herein, the NELL-1 is administered to the subject by injection at least one time per day over a treatment course from 1 day to about 365 days.

In some embodiments of the invention method, optionally in combination with any of the various embodiments disclosed herein, the NELL-1 protein or peptide is exogenic NELL-1 protein or peptide.

In some embodiments of the invention method, optionally in combination with any of the various embodiments disclosed herein, the NELL-1 protein or peptide is administered by a recombinantly engineered cell expressing the NELL-1 protein or peptide.

In some embodiments of the invention method, optionally in combination with any of the various embodiments disclosed herein, the osteoinflammation preventive agent further comprises a non-steroidal anti-inflammatory drug (NSAID).

In some embodiments of the invention method, optionally in combination with any of the various embodiments disclosed herein, the NELL-1 protein or peptide is provided by a transdermal delivery system.

In some embodiments of the invention method, optionally in combination with any of the various embodiments disclosed herein, the subject is a human being.

In one aspect of the present invention, it is provided a composition for cartilage arthritis in a subject, comprising an effective amount of an osteoinflammation preventive agent for cartilage arthritis, the osteoinflammation preventive agent comprising NELL-1 protein or peptide optionally in combination with a pro-chondrogenic agent.

In some embodiments of the invention composition, optionally in combination with any of the various embodiments disclosed herein, the osteoinflammation preventive agent further comprises a non-steroidal anti-inflammatory drug (NSAID).

In some embodiments of the invention composition, optionally in combination with any of the various embodiments disclosed herein, the NELL-1 protein or peptide is in an amount effective for anti-osteoinflammation.

In some embodiments of the invention composition, optionally in combination with any of the various embodiments disclosed herein, the composition comprises the pro-chondrogenic agent, wherein the pro-chondrogenic agent comprises the NELL-1 protein or peptide.

In some embodiments of the invention composition, optionally in combination with any of the various embodiments disclosed herein, the articular cartilage arthritis is osteoarthritis.

In some embodiments of the invention composition, optionally in combination with any of the various embodiments disclosed herein, the composition is in a formulation for injection.

In some embodiments of the invention composition, optionally in combination with any of the various embodiments disclosed herein, the composition is in a formulation for transdermal delivery system.

In some embodiments of the invention composition, optionally in combination with any of the various embodiments disclosed herein, the subject is a human being.

In one aspect of the present invention, it is provided a method of forming a composition, comprising:

providing an effective amount of an osteoinflammation preventive agent for cartilage arthritis, the osteoinflammation preventive agent comprising NELL-1 protein or peptide optionally in combination with a pro-chondrogenic agent, and forming the composition.

In some embodiments of the invention method, optionally in combination with any of the various embodiments disclosed herein, the osteoinflammation preventive agent further comprises a non-steroidal anti-inflammatory drug (NSAID).

In some embodiments of the invention method, optionally in combination with any of the various embodiments disclosed herein, the NELL-1 protein or peptide is in an amount effective for anti-osteoinflammation.

In some embodiments of the invention composition, optionally in combination with any of the various embodiments disclosed herein, the composition comprises the pro-chondrogenic agent, wherein the pro-chondrogenic agent comprises the NELL-1 protein or peptide.

In some embodiments of the invention method, optionally in combination with any of the various embodiments disclosed herein, the articular cartilage arthritis is osteoarthritis.

In some embodiments of the invention method, optionally in combination with any of the various embodiments disclosed herein, the composition is in a formulation for injection.

In some embodiments of the invention method, optionally in combination with any of the various embodiments disclosed herein, the composition is in a formulation for transdermal delivery system.

In some embodiments of the invention method, optionally in combination with any of the various embodiments disclosed herein, the subject is a human being.

### BRIEF DESCRIPTION OF DRAWINGS

FIG. 1 shows schematic depicting the intra-articular injection animal model. ‘Control’ group: 6 μl PBS per injection for 14 days; ‘NELL-1’ group: 6 μl PBS per injection for 7 days followed by 2 μg recombinant human NELL-1 in 6 μl PBS per injection for the next 7 days; ‘IL1β’ group: 100 ng recombinant human IL1β in 6 μl PBS per injection for 14 days; and ‘IL1β+NELL-1’ group: 100 ng IL1β in 6 μl PBS per injection for 7 days to trigger inflammation, and 100 ng IL1β+2 μg NELL-1 in 6 μl PBS per injection for the next 7 days. All injections were performed twice daily.

FIG. 2 shows characterization of WT and Nell-1-haploinsufficient (Nell-1^{+/6R}) mouse knee joints at 3 and 18 months of age. Representative photos of 3-(A), and 18-month-(B) old female WT and Nell-1^{+/6R} mouse knee joints. H&E staining was performed for histological analysis, while safranin 0 was used to stain proteoglycans. Expression of anabolic marker type II collagen (Collagen, type II), catabolic marker Mmp13, as well as proinflammatory markers interleukin (Il)1β and Il6 was evaluated by IF staining. DAPI was used for nuclear counterstaining. HC: uncalcified hyaline zone of articular cartilage; CC: calcified zone of articular cartilage. Solid arrows indicate the erosion in HC; open triangles indicate severe loss of HC. Bar=500 μm. Relative RNA expression in the tibial cartilage is presented in FIG. 13.

FIG. 3 shows characterization of mouse knee joints after 14 days of intra-articular injections. Representative photos of 2.5-month-old female WT (A) and Nell-1^{+/6R} (B) mouse knee joints after 14 days of intra-articular injections (3 months old at the end of treatment). The injection schematic of each group is presented in FIG. 1. H &E staining was performed for histological analysis, while safranin O was used to stain proteoglycans. Expression of Collagen, type II, Mmp13, Il1, and Il6 was evaluated by IF staining. DAPI was used for nuclear counterstaining. HC: uncalcified hyaline zone of articular cartilage; CC: calcified zone of articular cartilage. Relative RNA expression in the tibial cartilage is presented in FIG. 15, while gait scores are summarized in FIG. 16. Bar=500 μm.

FIG. 4 shows effects of NELL-1 on gene expression in primary mouse articular chondrocytes (mARCs). Expression of Il1β (A-A2), Il6 (B-B2), Tnfα (C-C2), Mmp13 (D-D2), Adamts5 (E-E2), Ptgs2 (F-F2), Col2α1 (G-G2), and Nell-1 (H-H2) was quantified by real-time PCR, and the data were normalized to the respective levels of WT-mARCs (A-H and A1-H1) or Nell-1^{+/6R}-mARCs (A2-H2) before treatment (dashed lines). Mean+SD of three independent experiments performed in duplicate are shown. One-way ANOVA and two-sample t-test analyses were performed. *, P<0.05, a suggestive difference; **, P<0.005, a statistically significant difference.

FIG. 5 shows effects of NELL-1 on gene expression in primary human articular chondrocytes (hARCs). Expression of IL1β (A-A3), IL6 (B-B3), TNFα (C-C3), MMP13 (D-D3), ADAMTS5 (E-E3), PTGS2 (F-F3), COL2α1 (G-G3), and NELL-1 (H-H3) was quantified by real-time PCR, and the data were normalized to the respective levels of pathological normal/health (NM)-hARCs (A-H and A1-H1), osteoarthritis (OA)-hARCs (A2-H2), or rheumatoid arthritis (RA)-hARCs (A3-H3) before treatment (dashed lines). Mean+SD of three independent experiments performed in duplicate are shown. One-way ANOVA and two-sample t-test analyses were performed. *, P<0.05, a suggestive difference; **, P<0.005, a statistically significant difference.

FIG. 6 shows effects of Nfatc1- and Runx1-KD on NELL-1's anti-inflammatory potency. Stable scramble, Nfatc1-, and Runx1-KD ATDC5 clones were established (FIG. 21). Expression of proinflammatory genes Il1β (A-A3), Il6 (B-B3), and Tnfα (C-C3) was quantified by real-time PCR, and the data were normalized to the respective levels of ATDC5 (A-C), ATDC5 (scramble) (A1-C1), ATDC5 (Nfatc1-KD) (A2-C2), or ATDC5 (Runx1-KD) (A3-C3) cells before treatment (dashed lines). Mean+SD of three independent experiments performed in duplicate are shown. One-way ANOVA and two-sample t-test analyses were performed. N.D.: not detectable. * P<0.05, a suggestive difference; **, P<0.005, a statistically significant difference.

FIG. 7 shows expression of Runx1 in mouse knees with intra-articular NELL-1 administration. Using IF, Runx1 expression was observed in 2.5-month-old female WT (A) and Nell-1^{+/6R} (B) mouse knee joints after 14 days of intra-articular injections (3 months old at the end of treatment). DAPI was used for nuclear counterstaining. HC: uncalcified hyaline zone of articular cartilage; CC: calcified zone of articular cartilage. Bar=500 μm.

FIG. 8 shows schematic depicting NELL-1's effects in articular cartilage. (A) Focal wear and tear of HC with early chondrocyte clustering became evident in the tibial plateau cartilage of 3-month-old Nell-1^{+/6R} mice, while severe loss of HC was observed in the knees of 18-month-old Nell-1^{+/6R} mice. (B) Our previous studies revealed that the NELL-1→NFATc1→RUNX3→IHH cascade in chondrocytes is responsible for NELL-1's pro-chondrogenic bioactivities. Here, we demonstrate that RUNX1, instead of NFATc1, is essential for NELL-1 to exhibit its anti-inflammatory properties in chondrocytes.

FIG. 9 shows Nell-1 expression in knee joints of WT and Nell-1^{+/6R} neonatal mice. Representative photos of neonatal female WT and Nell-1^{+/6R} mouse knee joints. Safranin O staining was used to identify the cartilage region. Bar=50 μm.

FIG. 10 shows expression levels of Nell-1 in medial tibial plateau cartilage of WT and Nell-1^{+/6R} mouse knees at different developmental stages. Tibial cartilage tissue was collected at 1-(A), 3-(B), and 18-months (C) from paraffin-embedded sections using LCM, and total RNA was isolated by the RNeasy© FFPE Kit. Data were normalized to the respective levels of 1-month-old WT mice (dashed lines). Mean+SD of three RNA pools (two animals per pool) performed in duplicate are shown. One-way ANOVA and two-sample t-test analyses were performed. *, P<0.05, a suggestive difference; **, P<0.005, a statistically significant difference.

FIG. 11 shows dissecting medial tibial plateau cartilage from the paraffin-embedded sections of mouse knee joints. Using the Cresyl Fast Violet staining, LCM permitted the excision of the tibial cartilage.

FIG. 12 shows characterization of WT and Nell-1^{+/6R} mouse knee joints at 1 month of age. H&E staining was performed for histological analysis, while safranin O was used to stain proteoglycans. Expression of anabolic marker type II collagen (Collagen, type II), catabolic marker Mmp13, and proinflammatory markers interleukin (Il)1β and Il6 was evaluated by IF staining, respectively. DAPI was used for nuclear counterstaining. HC: uncalcified hyaline zone of articular cartilage; CC: calcified zone of articular cartilage. Bar=500 μm.

FIG. 13 shows transcription levels of Il1β, Il6, Mmp13, and Col2α1 in tibial cartilage of WT and Nell-1^{+/6R} mouse knees. RNA levels of 111 (A), Il6 (B), Mmp13 (C), and Col2α1 (D) in the tibial cartilage of WT and Nell-1^{+/6R} mouse knees were tracked by quantitative real-time PCR. Data were normalized to the respective levels of 1-month-old WT mice (dashed lines). Mean+SD of three RNA pools (two animals per pool) performed in duplicate are shown. One-way ANOVA and two-sample t-test analyses were performed. *, P<0.05, a suggestive difference; **, P<0.005, a statistically significant difference.

FIG. 14 shows characterization of WT and Nell-1^{+/6R} mouse knee joints after 7 days of IL10 intra-articular injections. Representative photos of 2.5-months-old female mouse knee joints after 7 days of IL1β intra-articular injections. One hundred ng recombinant human IL10 in 6 μl PBS was administered in each injection, and the injections were given twice daily for 7 days. H&E staining was performed for histological analysis, while safranin O was used to stain proteoglycans. Expression of Collagen, type II, Mmp13, 111, and Il6 was evaluated by IF staining. DAPI was used for nuclear counterstaining. HC: uncalcified hyaline zone of articular cartilage; CC: calcified zone of articular cartilage. Bar=500 μm.

FIG. 15 shows RNA levels of Il1β, Il6, Mmp13, Col2α1, Nell-1, and Runx1 in tibial cartilage of WT and Nell-1^{+/6R} mouse knees after 14 days of intra-articular injections. The intra-articular injections were performed for 14 days in both WT and Nell-1^{+/6R} mice, as described in FIG. 1. RNA levels of Il1β (A), Il6 (B), Mmp13 (C), Col2α1 (D), Nell-1 (E), and Runx1 (F) in the tibial cartilage of WT and Nell-1^{+/6R} mice were tracked by quantitative real-time PCR. Data were normalized to the respective levels of control WT mice (dashed lines). Mean+SD of three RNA pools (two animals per pool) performed in duplicate are shown. One-way ANOVA and two-sample t-test analyses were performed. *, P<0.05, a suggestive difference; **, P<0.005, a statistically significant difference.

FIG. 16 shows observational gait scoring of WT and Nell-1^{+/6R} mice after intra-articular injections. After 14 days of intra-articular injections (FIG. 1), gait scores of WT (A) and Nell-1^{+/6R} (B) mice were determined by three independent and experienced physicians in a blinded fashion. (C) In a validation experiment, the gait scores were given to Nell-1^{+/6R} mice after 7 and 14 days intra-articular injections (FIG. 1). For each animal, average scores from the three physicians and the mean value of each group (N=6 animals) are presented. Kruskal-Wallis ANOVA (A), the Mann-Whiney U test (B), and the paired-sample Wilcoxon test (C) were used for statistical analyses, respectively. *, P<0.05, a suggestive difference; **, P<0.005, a statistically significant difference.

FIG. 17 shows high IL1β staining was accompanied by low NELL-1 levels in human arthritic articular cartilage lesions. Representative photos of human articular cartilage samples with various degrees of arthritis severity. H&E staining was performed for histological analysis, while safranin O was used to observe arthritis severity by staining proteoglycans. Expression of IL1β, NELL-1, BMP6, and BMP7 was elucidated by IF staining. Magenta (in safranin O images, except for Donor 1, Sample 1), white (in the safranin O image of Donor 1, Sample 1), or red (in IF images) dotted lines outline the regions of high IL10 expression in articular cartilage, while black (in safranin O images) or yellow (in IF images) dotted lines outline the regions of high NELL-1 expression in articular cartilage. Bar=500 μm.

FIG. 18 shows anti-inflammatory effects of NELL-1 in primary mARCs. Secreted Il6 (A), Tnfα (B), and Mmp13 (C) were measured by ELISA after 6 h of treatment. Mean+SD of three independent samples assessed in duplicate are shown. One-way ANOVA and two-sample t-test analyses were performed. *, P<0.05, a suggestive difference; **, P<0.005, a statistically significant difference.

FIG. 19 shows anti-inflammatory effects of NELL-1 in primary hARCs. Secreted IL6 (A), TNFα (B), and MMP13 (C) were measured by ELISA after 6 h of treatment. Mean+SD of three independent samples assessed in duplicate are shown. One-way ANOVA and two-sample t-test analyses were performed. *, P<0.05, a suggestive difference; **, P<0.005, a statistically significant difference.

FIG. 20 shows effects of NELL-1 on the gene expression in ATDC5 cells. Expression of Il1β (A), Il6 (B), Tnfα (C), Mmp13 (D), Adamts5 (E), and Ptgs2 (F) was quantified by real-time PCR, and the data were normalized to the respective levels before treatment (dashed lines). Mean+SD of three independent experiments performed in duplicate are shown. One-way ANOVA and two-sample t-test analyses were performed. N.D.: not detectable. *, P<0.05, a suggestive difference; **, P<0.005, a statistically significant difference.

FIG. 21 shows establishment of stable Nfatc1- and Runx1-KD ATDC5 clones. Expression of Nfatc1 (A) and Runx1 (B) was quantified by real-time PCR, and the data were normalized to the respective levels of ATDC5 cells before treatment (dashed lines). Mean+SD of three independent experiments performed in duplicate are shown. One-way ANOVA and two-sample t-test analyses were performed. *, P<0.05, a suggestive difference; **, P<0.005, a statistically significant difference.

FIG. 22 shows effects of Nfatc1- and Runx1-KD on NELL-1's anti-inflammatory activity in ATDC5 cells. Secreted Il6 (A) and Tnfα (B) were measured by ELISA after 6 h of treatment. Mean+SD of three independent samples assessed in duplicate are shown. One-way ANOVA and two-sample t-test analyses were performed. *, P<0.05, a suggestive difference; **, P<0.005, a statistically significant difference.

FIG. 23 shows Runx1 expression in ATDC5 cells. Expression of Runx1 in non-transfected ATDC5 (A), scramble ATDC5 (B), and Runx1-KD ATDC5 cells (C) was qualified by real-time PCR, and the data were normalized to the levels of non-transfected ATDC5 cells before treatment (dashed lines). Mean+SD of three independent experiments performed in duplicate are shown. One-way ANOVA and two-sample t-test analyses were performed. *, P<0.05, a suggestive difference; **, P<0.005, a statistically significant difference.

FIG. 24 shows Runx1 expression in primary mARCs. Expression of Runx1 was quantified by real-time PCR, and the data were normalized to the respective levels of WT-mARCs (A and B) or Nell-1^{+/6R}-mARCs (C) before treatment (dashed lines). Mean+SD of three independent experiments performed in duplicate are shown. One-way ANOVA and two-sample t-test analyses were performed. *, P<0.05, a suggestive difference; **, P<0.005, a statistically significant difference.

FIG. 25 shows RUNX1 expression in primary hARCs. Expression of RUNX1 was quantified by real-time PCR, and the data were normalized to the respective levels of NM-hARCs (A and B), OA-hARCs (C), or RA-hARCs (D) before treatment (dashed lines). Mean+SD of three independent experiments performed in duplicate are shown. One-way ANOVA and two-sample t-test analyses were performed. *, P<0.05, a suggestive difference; **, P<0.005, a statistically significant difference.

### DETAILED DESCRIPTION OF EMBODIMENTS OF THE INVENTION

### Definitions

Osteoarthritis: Traditionally, osteoarthritis (“OA”) was viewed as an inevitably progressive, degenerative disease process. New work suggests that it is a dynamic process that may progress episodically. It is a heterogeneous group of diseases characterized by an adaptive response of synovial joints to a variety of environmental, genetic, and biomechanical stresses. While OA is not caused by inflammation, OA can still result in some inflammation of the joints. The difference is that this inflammation probably results from wear and tear. This is in contrast with rheumatoid arthritis (“RA”), which is an autoimmune disease in which the body's immune system mistakenly attacks the joints. NSADs—nonsteroida anti-inflammatory drugs are commonly used to treat RA.

As used herein, the term “pro-chondrogenic agent” refers to an agent that is effective in promoting cartilage regeneration. Some examples are such pro-chondrogenic agents are provided below in Table 7,

TABLE 7

| Biological | | |
|---|---|---|
| Process | Mediators | Potential Therapeutics*^{/}** |
| Autophagy and | PPARγ [87, 178, 179] | mTOR [85, 98]; AKT/Fox03/mTOR [99]; |
| cell survival | CXCR2 [•180] | Rapamycin, polyamines, ω-6 polyunsaturated |
| | | fatty acids; glucosamine |
| | | Chemokine antagonists orblocking antibodies |
| Chondrogenesis | Runx1 | TD-198946 [181]; Kartogenin [182, 183] |
| and inhibition | PTH receptor | Recombinant humanPTH (1-34) |
| of endochondral | EGFR signaling | (teriparatide)[184] |
| ossification | | Mitogen-inducible gene 6 (MIG6) [185] |
| Calcification | NPP1 [861] | Phosphocitrates, TLR and NLRP3 inhibition |
| and crystals | transglutaminase, inorganic | |
| | pyrophosphate, TLRs, | |
| | NLRP3 | |
| Subchondral | WNT/β-catenin | Wnt antagonists (DKK1, SOST) |
| bone | BMPs | BMP antagonists (Gremlin, follistatin) [136- |
| | BMP-7 | 138, 141] |
| | TGF-β [117] | Recombinant BMP-7 (Eptotermin) [187] |
| | | TGF-β-specific antibody |
| Cartilage | IGF-1 | Humanized IGF-1-heparin-binding domain |
| anabolism | FGF-18 | fusion protein.[190] |
| | PRG4 [••61] | Sprifermin [133] |
| | NFATc2/c2[188] | Oral or intraarticular calcitonin [191-193] |
| | Calcitonin [1891 | |
| Circadian clock | Bmal1 [•194] | REV-ERB agonists [195] |

*Both pre-clinical and clinical studies referred to without comment on efficacy.
**See Mary B. Goldring and Francis Brenbaum; Curr Opin Pharmacol. 2015 June; 22: 51-63, Table 2; reference numbers are as provided therein.

As used herein, the term “osteoinflammation preventive agent” refers to an agent that prevents osteoinflammation or significantly reduces or decreases osteoinflammation in a subject, e.g., reduction of osteoinflammation by a factor of from about 20% to about 100%, about 30% to about 100%, about 40% to about 100%, about 50% to about 100%, about 60% to about 100%, about 70% to about 100%, about 80% to about 100%, about 90% to about 100%.

As used herein, the term “NELL (Nel-like molecule-1; Nel (a protein strongly expressed in neural tissue encoding epidermal growth factor like domain)) peptides” can be NELL1 or NELL2 polypeptide, or a fragment thereof (SEQ ID NO: 1 and SEQ ID NO: 2 respectively); a NELL1 or NELL2 related polypeptide, or a fragment thereof, any polypeptide with significant homology to “NELL-1 protein or peptides” or a fragment thereof. Significant homology can be construed to mean >50% homology to “NELL-1 protein or peptides”, e.g., >60% homology to “NELL-1 protein or peptides”, >70% homology to “NELL-1 protein or peptides,” or >80% homology to “NELL-1 protein or peptides.” The NELL-1 protein or peptides can be natural and/or recombinant NELL-1 protein or peptides with a non-mutated wild-type sequence or recombinant NELL-1 protein or peptides with a mutated wild-type sequence that still contains significant homology to NELL-1 protein or peptides. In addition, NELL-1 protein or peptides can be derived from, but not limited to, an organism such as human cells, bacteria, yeast, or insect or plant cells. In some embodiments, the term “NELL-1 protein or peptide” includes structural, functional or conformational equivalents of NELL-1 protein or peptide. As used herein, a structural equivalent of a NELL-1 protein or peptide refers to a protein or peptide including a structure equivalent or substantially similar to that of a NELL-1 protein or peptide or of a functional domain of a NELL-1 protein or peptide. A functional equivalent of a NELL-1 protein or peptide refers to a protein or peptide having a function equivalent or substantially similar to that of a NELL-1 protein or peptide or of a functional domain of a NELL-1 protein or peptide. A conformational equivalent of a NELL-1 protein or peptide refers to a protein or peptide having a conformation equivalent or substantially similar to that of a NELL-1 protein or peptide or of a functional domain of a NELL-1 protein or peptide.

In some embodiments, the NELL-1 protein or peptide described herein can be a derivative of the NELL-1 protein or peptide. The term “derivative” as used herein, refers to any chemical or biological compounds or materials derived from a NELL-1 protein or peptide, structural equivalents thereof, or conformational equivalents thereof. For example, such a derivative can include any pro-drug form, PEGylated form, or any other form of a NELL-1 protein or peptide that renders the NELL-1 protein or peptide more stable or to have a better osteophilicity or lipophilicity. In some embodiments, the derivative can be a NELL-1 protein or peptide attached to poly(ethylene glycol), a poly(amino acid), a hydrocarbyl short chain having C1-C20 carbons, or a biocompatible polymer. In some embodiments, the term “derivative” can include a NELL-1 protein or peptide mimetics. Synthesis of mimetics of a peptide is well documented in the art. The following describes an example of the basic procedure for the synthesis of a peptide, including a peptide mimetics.

Before the peptide synthesis starts, the amine terminus of the amino acid (starting material) can be protected with FMOC (9-fluoromethyl carbamate) or other protective groups, and a solid support such as a Merrifield resin (free amines) is used as an initiator. Then, step (1) through step (3) reactions are performed and repeated until the desired peptide is obtained: (1) a free-amine is reacted with carboxyl terminus using carbodiimide chemistry, (2) the amino acid sequence is purified, and (3) the protecting group, e.g., the FMOC protecting group, is removed under mildly acidic conditions to yield a free amine. The peptide can then be cleaved from the resin to yield a free standing peptide or peptide mimetics.

In some embodiments, the peptide derivative described herein includes a physically or chemically modified NELL-1 protein or peptide. Physically modified peptide can be modification by, for example, modification by ionic force such as forming an ionic pair with a counterion, modification by hydrogen bonding, modification by modulation of pH, modulation by solvent selection, or modification by using different protein folding/unfolding procedures, which can involve selection of folding/unfolding temperature, pH, solvent, and duration at different stage of folding/unfolding.

In some embodiments, the peptide derivative can include a chemically modified NELL-1 protein or peptide. For example, a short hydrocarbon group(s) (e.g. methyl or ethyl) can be selectively attached to one or multiple sites on the NELL-1 protein or peptide molecule to modify the chemical and/or physical properties of the peptide. In some embodiments, a mono-, oligo- or poly(ethylene glycol) (PEG) group(s) can be selectively attached to one or multiple sites on the NELL-1 protein or peptide molecule to modify the chemical and/or physical properties of the peptide by commonly known protein PEGylation procedures (see, e.g., Mok, H., et al., Mol. Ther., 11(1):66-79 (2005)).

The term “enhancer of NELL-1 protein or peptides” refers to a chemical or biological compound capable of enhancing the activity of NELL-1 protein or peptides. The term also includes a chemical or biological compound capable of enhancing the expression of NELL-1 protein or peptides. As examples, methods of interactions can include but are not limited to increased transcription or translation of NELL-1 protein or peptides, increased stability of NELL-1 protein or peptide transcripts or protein products, increased activity of NELL-1 protein or peptide transcripts or protein products, and decreased degradation of NELL-1 protein or peptide transcript or protein products.

The term “modulator of NELL-1 protein or peptide receptors” refers to a chemical or biological compound capable of facilitating or inhibiting the binding of NELL-1 protein or peptide receptors to or by NELL-1 protein or peptides or to a chemical or biological compound capable of modulating NELL-1 protein or peptide receptor activity irrespective of the presence or the absence of NELL-1 protein or peptide. The modulator that facilitates the binding and/or activation of NELL-1 protein or peptide receptors to or by NELL-1 protein or peptides is referred to as an “agonist” of the receptor, and the modulator that inhibits the binding and/or activation of NELL-1 protein or peptide receptors to or by NELL-1 protein or peptides is referred to as an “antagonist” of the receptor. The modulator that facilitates the activation of NELL-1 protein or peptide receptors irrespective of NELL-1 protein or peptides is referred to as an “activator” of the receptor, and the modulator that inhibits activation of NELL-1 protein or peptide receptors irrespective of NELL-1 protein or peptides is referred to as an “inhibitor” of the receptor.

The term “NELL-1 protein or peptide”, “enhancer of NELL-1 protein or peptide” or “modulator of NELL-1 protein or peptide receptor(s)” is also referred to as an “agent” throughout the specification from time to time.

As used herein, the term “an effective amount” is meant to be an amount of NELL-1 protein or peptide to ameliorate or treat at least an osteoinflammatory cartilage condition in a subject, e.g., a mammal, producing a statistically significant therapeutic result in the subject.

### Articular Cartilage Degradation

Articular cartilage is comprised of mostly water (60-80 wt %) and the remaining ECM comprises mostly type II collagen (50-90% dry mass) and proteoglycans (5-10%). Other collagens and minor ECM molecules have been identified in small quantities. It is organization of the ECM into distinct zones, and the interaction between water and the ECM in the various zones that provide the toughness that is required for the absorption and transmission of biomechanical forces across joints, and simultaneously the frictionless articulating surfaces that are needed for joint motion. Stresses as high as 4 and 20 MPa have been reported in human hip joints during routine walking and jumping, respectively! As amazing as the articular cartilage is, it exhibits unfortunately minimal capacity for repair.

Over 20 million Americans suffer from osteoarthritis and degenerative joint diseases with an associated annual healthcare burden of over $60 billion. A wide array of scaffolds, cytokines, and growth factors have been investigated for cartilage tissue engineering (see, e.g., Frenkel, S. R., et al., Ann. Biomed. Eng. 32:26-34 (2004); Tuli, R., et al., Arthritis Res. Ther. 5:235-238 (2003); and Ashammakhi, N. and Reis, R L. Topics in Tissue Engineering, Vol. 2, 2005). The role of static vs. dynamic compression, shear stress, hydrostatic pressure, fluid flow, electrical streaming potentials, bioreactors, and complex loading on chondrocyte biological response and tissue remodeling have been investigated extensively and the mechanotransduction pathways reviewed Ashammakhi, N. and Reis, R L. Topics in Tissue Engineering, Vol. 2, 2005).

The various mechanisms in cartilage degradation of different stages of in osteoarthritis was reviewed by Elena V. Tchetina in Arthritis, Vol. 2011, pp 1-16 (Review) (2011). None of these identified a role by NELL-1 protein or peptide.

Cartilage is made of water (70%) and a type H collagen framework with proteoglycans and glycosaminoglycans (consisting mainly of aggrecan and also chondroitin), produced by chondrocytes. Proteoglycans in turn bind to hyaluronate which stabilizes the macromolecule. Chondrocytes receive nutrition from the synovium by diffusion and the synovial fluid is circulated by joint movement. It has been postulated that if the joint stops moving (as a result of a fracture or immobility) and chondrocytes lose their source of nutrition, they go into shock and cartilage repair ceases. Metalloproteinases are produced, which catalyze collagen and proteoglycan degradation. The synovium has been shown to be variably inflamed in osteoarthritis producing increased levels of interleukin-1 (IL-1) and tumor necrosis factor-alpha (TNF-α), cytokines that induce nitric oxide and metalloproteinase production. Interleukin-6 (IL-6) and mechanical loading of the joint also induce catabolic cytokine receptors. These bind IL-1 and TNF-α within cartilage causing more destruction.

### NELL-1 and Osteoarthritis

In human arthritis articular cartilage, inflammation is associated with reduced neural EGFL like 1 (NELL-1) expression. As a loss-of-function animal model, Nell-1-haploinsufficient (Nell-1^{+/6R}) mice have accelerated and aggravated osteoarthritis progression accompanied with elevated inflammatory markers. NELL-1 strongly suppressed the expression of inflammatory cytokines as well as their downstream catabolic enzymes that are responsible for cartilage extracellular matrix degradation in mouse and human articular cartilage chondrocytes. Moreover, NELL-1 administration significantly reduced the inflammatory reaction and articular cartilage damages in vivo. Excitingly, the inflammatory induced heavy ‘lame-walking’ and ‘fear-to-walk’ symptoms observed in Nell-1^{+/6R} mice were significantly recovered by the NELL-1 application.

By examining human arthritis cartilage samples as well as animal models simulating arthritis, we demonstrate the emerging role of NELL-1 in arthritis pathogenesis and identify NELL-1 as a promising anti-osteoinflammation agent for preventing and suppressing arthritis-related cartilage damages. Thus this invention provides the fundamental of clinical application of NELL-1 as a disease-modifying anti-arthritis agent.

### Enhancers of NELL-1 Protein or Peptides

In another embodiment, it is provided an osteoinflammation preventive composition that includes one or more enhancers of NELL-1 protein or peptides.

### Modulators of Receptors of NELL-1 Protein or Peptides

In a further aspect of the present invention, the osteoinflammation preventive composition provided herein can include a modulator of a receptor of NELL-1 protein or peptide. NELL1 and NELL2 proteins are secretory molecules which bind to membrane bound receptors (Kuroda, S., et al., Biochem Biophys Res Commun, 265(1): p. 79-86) (1999).

Modulators of the receptors of NELL-1 protein or peptides can be identified by any established method for screening for modulators of a receptor. In one embodiment, the modulators of the receptors of NELL-1 protein or peptides can be screened for by competitive binding. For example, one method of screening for such modulators can include the following steps: (1) contacting a receptor molecule of a NELL-1 protein or peptide with a test compound, (2) contacting the NELL-1 protein or peptide with the receptor molecule and the test compound, (3) detecting the extent of binding of the NELL-1 protein or peptide to the receptor molecule with the test compound, (4) comparing the extent of binding of the NELL-1 protein or peptide to the receptor molecule with the test compound with the extent of binding of a control wherein the control is obtained by detecting the extent of binding of the NELL-1 protein or peptide to the receptor molecule without the test compound, and (5) designating the test compound as a modulator of the receptor of the NELL-1 protein or peptide if the extent of binding of the NELL-1 protein or peptide to the receptor molecule with the test compound is different from the extent of binding of the control. The modulators can be designated as an antagonist or an agonist of the receptor. If the extent of binding of the NELL-1 protein or peptide to the receptor molecule with the test compound is lower than the extent of binding of the control, the modulator is an antagonist of the receptor of the NELL-1 protein or peptide. If the extent of binding of the NELL-1 protein or peptide to the receptor molecule with the test compound is higher than the extent of binding of the control, the modulator is designated as an agonist of the receptor of the NELL-1 protein or peptide.

In some embodiments, the NELL modulators described herein can include molecules that stabilize NELL and/or NELL receptors, as well as molecules that are involved in the stabilization and phosphorylation of the NELL-receptor complex after initial receptor ligation. In some embodiments, the modulators described herein can include agonists and antagonists of the aforementioned agonists and antagonists. In all cases, please expand the clinical applications to include those discussed in previous paragraph.

Modulators of a receptor of a NELL-1 protein or peptide can be screened for by manual testing or by an automated system such as a system based on combinatorial chemistry. One example of the screening system based on combinatorial chemistry is described in PCT/2003/029281 (WO 2004/024893).

### Osteoinflammation Preventive Composition

In one aspect of the present invention, it is provided a composition for cartilage arthritis in a subject, comprising an effective amount of an osteoinflammation preventive agent for cartilage arthritis, the osteoinflammation preventive agent comprising NELL-1 protein or peptide optionally in combination with a pro-chondrogenic agent.

In some embodiments of the invention composition, optionally in combination with any of the various embodiments disclosed herein, the osteoinflammation preventive agent further comprises a non-steroidal anti-inflammatory drug (NSAID).

In some embodiments of the invention composition, optionally in combination with any of the various embodiments disclosed herein, the NELL-1 protein or peptide is in an amount effective for anti-osteoinflammation.

In some embodiments of the invention composition, optionally in combination with any of the various embodiments disclosed herein, the composition comprises the pro-chondrogenic agent, wherein the pro-chondrogenic agent comprises the NELL-1 protein or peptide.

In some embodiments of the invention composition, optionally in combination with any of the various embodiments disclosed herein, the articular cartilage arthritis is osteoarthritis.

In some embodiments of the invention composition, optionally in combination with any of the various embodiments disclosed herein, the composition is in a formulation for injection.

In some embodiments of the invention composition, optionally in combination with any of the various embodiments disclosed herein, the composition is in a formulation for transdermal delivery system.

In some embodiments of the invention composition, optionally in combination with any of the various embodiments disclosed herein, the subject is a human being.

In a further aspect of the present invention, the osteoinflammation preventive composition provided herein includes at least a NELL-1 protein or peptide or an agonist of the receptor of NELL-1 protein or peptides in an amount effective for inducing chondroblast and chondrocyte to form cartilage. NELL proteins, peptides, DNA, RNA, and NELL agonists, and antagonist inhibitors can be used alone or in conjunction with scaffolds with and without cells, with or without mechanical stimulation, in the presence or absence of additional growth factors. For example, in one embodiment, the osteoinflammation preventive composition can be effective in regenerate cartilage in intervertebral disc, articular cartilage repair and regeneration. In another embodiment, the osteoinflammation preventive composition can be effective in forming cartilage via ex vivo gene therapy and protein application to cells with or without scaffold in tissue engineering.

Depending on the delivery method and the local environment, a composition including a NELL-1 protein or peptide can be used to induce an osteogenic cell, as such as a chondrocyte or chondroblast, to differentiate and form cartilage only. For example, in an articular cartilage defect, the composition described herein can induce an osteogenic cell such as chondrocyte/blast to form cartilage only. In some embodiments, the composition can optionally include cells (stem cells, chondroblast etc). In some embodiments, the composition can be applied as gene therapy.

Therefore, in some embodiments, the composition described herein can be used to regenerate/repair cartilage, e.g., for disc repair in articular cartilage and intervertebral disc.

Other exemplary cartilage conditions that can be treated, prevented, or ameliorated by an osteoinflammation preventive composition disclosed herein include, but are not limited to, chondrocalcinosis, osteoarthritis, and/or other diseases characterized by pathological cartilage degeneration.

### Other Agents

In some embodiments, the osteoinflammation preventive composition described herein may include a NELL-1 protein or peptide and another agent. Such other agents include, e.g., one of non-steroidal anti-inflammatory drugs (NSAIDs). Examples of NSAIDs are listed below in Table 1.

TABLE 1
Common non-steroidal anti-inflammatory drugs

| 1. Drug | 2. Commercial name |
|---|---|
| Aspirin (acetylsalicylic acid) | Bayer, Ecotrin |
| Celecoxib | Celebrex |
| Choline Magnesium Trisalicylate | Tricosal*, Trilisate* |
| Diclofenac | Arthrotec (combination product), Voltaren, Cataflam*, |
| | Cambia, Solaraze, Zipsor, Zorvolex, Flector, Pennsaid |
| Diflunisal | Dolobid |
| Etodolac | Lodine* |
| Fenoprofen calcium | |
| Flurbiprofen | Ansaid |
| Ibuprofen | Advil, Duexis (combination product), Motrin |
| Indomethacin | Indocin, Tivorbex |
| Ketoprofen | Orudis*, Oruvail*, Actron* |
| Ketorolac | Toradol*, SPRIX |
| Meclofenamate | Meclomen* |
| Mefenamic acid | |
| Meloxicam | Mobic |
| Nabumetone | Relafen* |
| Naproxen/naproxen sodium | Aleve, Anaprox, Naprelan, Naprosyn, Vimovo (combination |
| Naproxen and esomeprazole magnesium | product) |
| Oxaprozin | Daypro |
| Piroxicam | Feldene |
| Salsalate | Disalsid |
| Sulindac | Clinoril* |
| Tolmetin | Tolectin* |

In some embodiments, NELL-1 can be used in further combination with an anesthetic agent such as Tylenol.

In some embodiments, the composition described herein can specifically exclude one or more of the above listed agents.

### Dosages and Dosing Regimen

Dosages of NELL-1 protein or peptides and other agents can be determined according to methods known in the art based on type of agent, the disease, and other factors such as age and gender.

In one embodiment, the dosage of NELL-1 protein or peptide for anti-osteoinflammation or anti-arthritis generally ranges from 0.001 pg/mm² to 1 pg/mm², or more preferably from 0.001 ng/mm² to 1 ng/mm², or more preferably from 0.001 pig/mm² to 1 pig/mm², or more preferably from 0.001 mg/mm² to 1 mg/mm², or more preferably from 0.001 g/mm² to 1 g/mm², with or without a particular carrier or scaffold. In another embodiment, the dosage of NELL-1 protein or peptide for anti-osteoinflammation or anti-arthritis generally ranges from 0.001 pg/ml to 1 pg/ml, or more preferably from 0.001 ng/ml to 1 ng/ml, or more preferably from 0.001 pg/ml to 1 μg/ml, or more preferably from 0.001 mg/ml to 1 mg/ml, or more preferably from 0.001 g/ml to 100 g/ml, with or without a particular carrier or scaffold. In yet another embodiment, the dosage of NELL-1 protein or peptide for anti-osteoinflammation or anti-arthritis generally ranges from 0.001 pg/kg to 1 pg/kg, or more preferably from 0.001 ng/kg to 1 ng/kg, or more preferably from 0.001 μg/kg to 1 μg/kg, or more preferably from 0.001 mg/kg to 1 mg/kg, or more preferably from 0.001 gm/kg to 1 gm/kg, more preferably from 0.001 kg/kg to 1 kg/kg with or without a particular carrier or scaffold. Furthermore, it is understood that all dosages may be continuously given or divided into dosages given per a given timeframe. Examples of timeframes include but are not limited to every 1 hour, 2 hours, 4 hours, 6 hours, 8 hours, 12 hours, 24 hours, 48 hours, or 72 hours, or every week, 2 weeks, 4 weeks, or every month, 2 months, 4 months, and so forth.

In one embodiment, the even more preferable optimal dosage ranges of NELL-1 protein or peptides include but are not limited to 1 ng/mm² to 100 ng/mm², or even more preferably from 100 ng/mm² to 1000 ng/mm², or even more preferably from 1 μg/mm² to 100 μg/mm², or even more preferably from 100 μg/mm² to 1000 μg/mm². In another embodiment, the even more preferable optimal dosage ranges of NELL-1 protein or peptides include but are not limited to 1 ng/ml to 100 ng/ml, or even more preferably from 100 ng/ml to 1000 ng/ml, or even more preferably from 1 μg/ml to 100 μg/ml, or even more preferably from 100 μg/ml to 1000 μg/ml. In yet another embodiment, even more preferable optimal dosage ranges of NELL-1 protein or peptide for anti-osteoinflammation or anti-arthritis generally range from 1 μg/kg to 100 μg/kg, or even more preferably from 100 μg/kg to 1000 μg/kg, or even more preferably from 1 mg/kg to 100 mg/kg with or without a particular carrier or scaffold. Furthermore, it is understood that all dosages may be continuously given or divided into dosages given per a given timeframe. Examples of timeframes include but are not limited to every 1 hour, 2 hours, 4 hours, 6 hours, 8 hours, 12 hours, 24 hours, 48 hours, or 72 hours, or every week, 2 weeks, 4 weeks, or every month, 2 months, 4 months, and so forth. As used herein, the term “significantly above the optimal range” means, e.g., about 1% to about 50%, about 5% to about 50%, about 10% to about 50%, about 20% to about 50%, about 30% to about 50%, or about 40% to 50% over the optimal range.

The dosage for inhibitors of NELL-1 protein or peptides varies according to the type of the inhibitor, the cartilage condition to be treated, prevented, or ameliorated, and the age, the location, and the gender of the mammalian subject receiving the osteoinflammation preventive composition containing the inhibitor. Generally, the dosage for inhibitors of NELL-1 protein or peptides ranges from but is not limited to: 0.001 pg/mm² to 1 pg/mm², or more preferably from 0.001 ng/mm² to 1 ng/mm², or more preferably from 0.001 μg/mm² to 1 μg/mm², or more preferably from 0.001 mg/mm² to 1 mg/mm², or more preferably from 0.001 g/mm² to 1 g/mm², with or without a particular carrier or scaffold. In another embodiment, the dosage for inhibitors of NELL-1 protein or peptides generally ranges from 0.001 pg/ml to 1 pg/ml, or more preferably from 0.001 ng/ml to 1 ng/ml, or more preferably from 0.001 μg/ml to 1 μg/ml, or more preferably from 0.001 mg/ml to 1 mg/ml, or more preferably from 0.001 g/ml to 100 g/ml, with or without a particular carrier or scaffold. In yet another embodiment, the dosage for inhibitors of NELL-1 protein or peptides generally ranges from 0.001 pg/kg to 1 pg/kg, or more preferably from 0.001 ng/kg to 1 ng/kg, or more preferably from 0.001 μg/kg to 1 μg/kg, or more preferably from 0.001 mg/kg to 1 mg/kg, or more preferably from 0.001 gm/kg to 1 gm/kg, more preferably from 0.001 kg/kg to 1 kg/kg with or without a particular carrier or scaffold. Furthermore, it is understood that all dosages may be continuously given or divided into dosages given per a given timeframe. Examples of timeframes include but are not limited to every 1 hour, 2 hours, 4 hours, 6 hours, 8 hours, 12 hours, 24 hours, 48 hours, or 72 hours, or every week, 2 weeks, 4 weeks, or every month, 2 months, 4 months, and so forth.

The dosage for modulators of receptors of NELL-1 protein or peptides varies according to the type of the inhibitor, the type of receptor, the cartilage condition to be treated, prevented, or ameliorated, and the age, the location, and the gender of the mammalian subject receiving the osteoinflammation preventive composition containing the modulators of receptors of NELL-1 protein or peptides. Generally, the dosage for modulators of receptors of NELL-1 protein or peptides ranges from but is not limited to: 0.001 pg/mm² to 1 pg/mm², or more preferably from 0.001 ng/mm² to 1 ng/mm², or more preferably from 0.001 μg/mm² to 1 μg/mm², or more preferably from 0.001 mg/mm² to 1 mg/mm², or more preferably from 0.001 g/mm² to 1 g/mm², with or without a particular carrier or scaffold. In another embodiment, the dosage for modulators of receptors of NELL-1 protein or peptides generally ranges from 0.001 pg/ml to 1 pg/ml, or more preferably from 0.001 ng/ml to 1 ng/ml, or more preferably from 0.001 pg/ml to 1 pg/ml, or more preferably from 0.001 mg/ml to 1 mg/ml, or more preferably from 0.001 μg/ml to 100 μg/ml, with or without a particular carrier or scaffold. In yet another embodiment, the dosage for modulators of receptors of NELL-1 protein or peptides generally ranges from 0.001 pg/kg to 1 pg/kg, or more preferably from 0.001 ng/kg to 1 ng/kg, or more preferably from 0.001 μg/kg to 1 μg/kg, or more preferably from 0.001 mg/kg to 1 mg/kg, or more preferably from 0.001 gm/kg to 1 gm/kg, more preferably from 0.001 kg/kg to 1 kg/kg with or without a particular carrier or scaffold. Furthermore, it is understood that all dosages may be continuously given or divided into dosages given per a given timeframe. Examples of timeframes include but are not limited to every 1 hour, 2 hours, 4 hours, 6 hours, 8 hours, 12 hours, 24 hours, 48 hours, or 72 hours, or every week, 2 weeks, 4 weeks, or every month, 2 months, 4 months, and so forth.

### Formulation Carriers

The osteoinflammation preventive composition described herein may be administered to a subject in need of treatment by a variety of routes of administration, including orally and parenterally, (e.g., intravenously, subcutaneously or intramedullary), intranasally, as a suppository or using a “flash” formulation, i.e., allowing the medication to dissolve in the mouth without the need to use water, topically, intradermally, subcutaneously and/or administration via mucosal routes in liquid or solid form. The osteoinflammation preventive composition can be formulated into a variety of dosage forms, e.g., extract, pills, tablets, microparticles, capsules, oral liquid.

There may also be included as part of the osteoinflammation preventive composition pharmaceutically compatible binding agents, and/or adjuvant materials. The active materials can also be mixed with other active materials including antibiotics, antifungals, other virucidals and immunostimulants which do not impair the desired action and/or supplement the desired action.

In one embodiment, the mode of administration of the osteoinflammation preventive composition described herein is oral. Oral compositions generally include an inert diluent or an edible carrier. They may be enclosed in gelatin capsules or compressed into tablets. For the purpose of oral therapeutic administration, the aforesaid compounds may be incorporated with excipients and used in the form of tablets, troches, capsules, elixirs, suspensions, syrups, wafers, chewing gums and the like. Some variation in dosage will necessarily occur, however, depending on the condition of the subject being treated. These preparations should produce a serum concentration of active ingredient of from about 0.01 nM to 1,000,000 nM, e.g., from about 0.2 to 40 μM. A preferred concentration range is from 0.2 to 20 μM and most preferably about 1 to 10 μM. However, the concentration of active ingredient in the drug composition itself depends on bioavailability of the drug and other factors known to those of skill in the art.

In another embodiment, the mode of administration of the osteoinflammation preventive compositions described herein is topical or mucosal administration. A specifically preferred mode of mucosal administration is administration via female genital tract. Another preferred mode of mucosal administration is rectal administration.

Various polymeric and/or non-polymeric materials can be used as adjuvants for enhancing mucoadhesiveness of the osteoinflammation preventive composition disclosed herein. The polymeric material suitable as adjuvants can be natural or synthetic polymers. Representative natural polymers include, for example, starch, chitosan, collagen, sugar, gelatin, pectin, alginate, karya gum, methylcellulose, carboxymethylcellulose, methylethylcellulose, and hydroxypropylcellulose. Representative synthetic polymers include, for example, poly(acrylic acid), tragacanth, poly(methyl vinylether-co-maleic anhydride), poly(ethylene oxide), carbopol, poly(vinyl pyrrolidine), poly(ethylene glycol), poly(vinyl alcohol), poly(hydroxyethylmethylacrylate), and polycarbophil. Other bioadhesive materials available in the art of drug formulation can also be used (see, for example, Bioadhesion—Possibilities and Future Trends, Gurny and Junginger, eds., 1990).

It is to be noted that dosage values also vary with the specific severity of the disease condition to be alleviated. It is to be further understood that for any particular subject, specific dosage regimens should be adjusted to the individual need and the professional judgment of the person administering or supervising the administration of the aforesaid compositions. It is to be further understood that the concentration ranges set forth herein are exemplary only and they do not limit the scope or practice of the invention. The active ingredient may be administered at once, or may be divided into a number of smaller doses to be administered at varying intervals of time.

The formulation may contain the following ingredients: a binder such as microcrystalline cellulose, gum tragacanth or gelatin; an excipient such as starch or lactose, a disintegrating agent such as alginic acid, Primogel, corn starch and the like; a lubricant such as magnesium stearate or Sterotes; a glidant such as colloidal silicon dioxide; and a sweetening agent such as sucrose or saccharin or flavoring agent such as peppermint, methyl salicylate, or orange flavoring may be added. When the dosage unit form is a capsule, it may contain, in addition to material of the above type, a liquid carrier such as a fatty oil. Other dosage unit forms may contain other various materials which modify the physical form of the dosage unit, for example, as coatings. Thus tablets or pills may be coated with sugar, shellac, or other enteric coating agents. Materials used in preparing these various compositions should be pharmaceutically pure and non-toxic in the amounts used.

The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methylparabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

The osteoinflammation preventive compositions of the present invention are prepared as formulations with pharmaceutically acceptable carriers. Preferred are those carriers that will protect the active compound against rapid elimination from the body, such as a controlled release formulation, including implants and microencapsulated delivery systems. Biodegradable, biocompatable polymers can be used, such as polyanhydrides, polyglycolic acid, collagen, and polylactic acid. Methods for preparation of such formulations can be readily performed by one skilled in the art.

Liposomal suspensions (including liposomes targeted to infected cells with monoclonal antibodies to viral antigens) are also preferred as pharmaceutically acceptable carriers. Methods for encapsulation or incorporation of compounds into liposomes are described by Cozzani, I.; Jori, G.; Bertoloni, G.; Milanesi, C.; Sicuro, T. Chem. Biol. Interact. 53, 131-143 (1985) and by Jori, G.; Tomio, L.; Reddi, E.; Rossi, E. Br. J. Cancer 48, 307-309 (1983). These may also be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

Other methods for encapsulating compounds within liposomes and targeting areas of the body are described by Sicuro, T.; Scarcelli, V.; Vigna, M. F.; Cozzani, I. Med. Biol. Environ. 15(1), 67-70 (1987) and Jori, G.; Reddi, E.; Cozzani, I.; Tomio, L. Br. J. Cancer, 53(5), 615-21 (1986).

The osteoinflammation preventive composition described herein may be administered in single (e.g., once daily) or multiple doses or via constant infusion. The compounds of this invention may also be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The osteoinflammation preventive compositions formed by combining the compounds of this invention and the pharmaceutically acceptable carriers, vehicles or diluents are then readily administered in a variety of dosage forms such as tablets, powders, lozenges, syrups, injectable solutions and the like. These osteoinflammation preventive compositions can, if desired, contain additional ingredients such as flavorings, binders, excipients and the like according to a specific dosage form.

Thus, for example, for purposes of oral administration, tablets containing various excipients such as sodium citrate, calcium carbonate and/or calcium phosphate may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the active pharmaceutical agent therein may be combined with various sweetening or flavoring agents, coloring matter or dyes and, if desired, emulsifying or suspending agents, together with diluents such as water, ethanol, propylene glycol, glycerin and/or combinations thereof.

For parenteral administration, solutions of the compounds of this invention in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of a compound of this invention. Capsules and cartridges (made, for example, from gelatin) for use in an inhaler or insufflator may be formulated containing a powder mix of a compound or compounds of the invention and a suitable powder base such as lactose or starch.

The osteoinflammation preventive composition described herein can be formulated alone or together with the other agent in a single dosage form or in a separate dosage form. Methods of preparing various pharmaceutical formulations with a certain amount of active ingredient are known, or will be apparent in light of this disclosure, to those skilled in this art. For examples of methods of preparing pharmaceutical formulations, see Remington's Pharmaceutical Sciences, Mack Publishing Company, Easton, Pa., 19th Edition (1995).

### Transdermal Delivery Formulation

In one embodiment, a transdermal delivery formulation can be formed, comprising an osteoinflammation preventive effective amount of NELL-1. In some embodiments, such topical formulation can optionally include a topical delivery patch, for example. Such topical delivery patch can include an element to effect transdermal delivery of a peptide or protein drug. Such element can include, for example, microneedles adapted to provide pores or micro-pores on skin of a joint or near the joint to allow transdermal delivery of NELL-1, and optionally, a second agent described above. Such element can also be a transdermal drug delivery formulation or device. An example of such device is described in U.S. patent application publication No. 2012/0220981 by Soo et al. Other examples are found in U.S. Pat. No. 5,356,632A to Gross et al, which describes a transdermal delivery device comprising a base member of insulating material; an anode electrode and a cathode electrode supported on said base member in spaced relation to each other to define a gap therebetween; means for connecting said electrodes to a voltage source; and an insulating layer releasably containing a liquid drug to be delivered covering said gap and both of said electrodes such that neither of said electrodes comes into contact with the subject's skin when applied thereto. The teachings in U.S. patent application publication No. 2012/0220981 and US5356632A are incorporated herein in their entirety.

In one embodiment, transdermal delivery formulation can include a carrier. The carrier may be biodegradable, such as degradable by enzymatic or hydrolytic mechanisms. Examples of carriers include, but are not limited to synthetic absorbable polymers such as but not limited to poly(a-hydroxy acids) such as poly (L-lactide) (PLLA), poly (D, L-lactide) (PDLLA), polyglycolide (PGA), poly (lactide-co-glycolide (PLGA), poly (-caprolactone), poly (trimethylene carbonate), poly (p-dioxanone), poly (-caprolactone-co-glycolide), poly (glycolide-co-trimethylene carbonate), poly (D, L-lactide-co-trimethylene carbonate), polyarylates, polyhydroxybutyrate (PHB), polyanhydrides, poly (anhydride-co-imide), propylene-co-fumarates, polylactones, polyesters, polycarbonates, polyanionic polymers, polyanhydrides, polyester-amides, poly(amino-acids), homopolypeptides, poly(phosphazenes), poly (glaxanone), polysaccharides, and poly(orthoesters), polyglactin, polyglactic acid, polyaldonic acid, polyacrylic acids, polyalkanoates; copolymers and admixtures thereof, and any derivatives and modifications. See for example, U.S. Pat. No. 4,563,489, and PCT Int. Appl. #WO/03024316, herein incorporated by reference. Other examples of carriers include cellulosic polymers such as, but not limited to alkylcellulose, hydroxyalkylcellulose, methylcellulose, ethylcellulose, hydroxyethylcellulose, hydroxypropylcellulose, hydroxypropyl-methylcellulose, carboxymethylcellulose, and their cationic salts. Other examples of carriers include synthetic and natural bioceramics such as, but not limited to calcium carbonates, calcium phosphates, apatites, bioactive glass materials, and coral-derived apatites. See for example U.S. Patent Application 2002187104; PCT Int. Appl. WO/9731661; and PCT Int. Appl. WO/0071083, herein incorporated by reference.

In one embodiment, the carrier may further be coated by compositions, including bioglass and/or apatites derived from sol-gel techniques, or from immersion techniques such as, but not limited to simulated body fluids with calcium and phosphate concentrations ranging from about 1.5 to 7-fold the natural serum concentration and adjusted by various means to solutions with pH range of about 2.8-7.8 at temperature from about 15-65 degrees C. See, for example, U.S. Pat. Nos. 6,426,114 and 6,013,591; and PCT Int. Appl. WO/9117965 herein incorporated by reference.

Other examples of carriers include collagen (e.g. Collastat, Helistat collagen sponges), hyaluronan, fibrin, chitosan, alginate, and gelatin, or a mixture thereof. See for example, PCT Int. Appls. WO/9505846; WO/02085422, the teachings of which are incorporated herein by reference.

In one embodiment, the carrier may include heparin-binding agents; including but not limited to heparin-like polymers e.g. dextran sulfate, chondroitin sulfate, heparin sulfate, fucan, alginate, or their derivatives; and peptide fragments with amino acid modifications to increase heparin affinity. See for example, Journal of Biological Chemistry (2003), 278(44), p. 43229-43235, the teachings of which are incorporated herein by reference.

In one embodiment, the substrate may be in the form of a liquid, solid or gel.

In one embodiment, the substrate may include a carrier that is in the form of a flowable gel. The gel may be selected so as to be injectable, such as via a syringe at the site where treatment is desired. The gel may be a chemical gel which may be a chemical gel formed by primary bonds, and controlled by pH, ionic groups, and/or solvent concentration. The gel may also be a physical gel which may be formed by secondary bonds and controlled by temperature and viscosity. Examples of gels include, but are not limited to, pluronics, gelatin, hyaluronan, collagen, polylactide-polyethylene glycol solutions and conjugates, chitosan, chitosan & P-glycerophosphate (BST-gel), alginates, agarose, hydroxypropyl cellulose, methyl cellulose, polyethylene oxide, polylactides/glycolides in N-methyl-2-pyrrolidone. See for example, Anatomical Record (2001), 263(4), 342-349, the teachings of which are incorporated herein by reference.

In one embodiment, the carrier may be photopolymerizable, such as by electromagnetic radiation with wavelength of at least about 250 nm. Example of photopolymerizable polymers include polyethylene (PEG) acrylate derivatives, PEG methacrylate derivatives, propylene fumarate-co-ethylene glycol, polyvinyl alcohol derivatives, PEG-co-poly(-hydroxy acid) diacrylate macromers, and modified polysaccharides such as hyaluronic acid derivatives and dextran methacrylate. See for example, U.S. Pat. No. 5,410,016, herein incorporated by reference.

In one embodiment, the substrate may include a carrier that is temperature sensitive. Examples include carriers made from N-isopropylacrylamide (NiPAM), or modified NiPAM with lowered lower critical solution temperature (LCST) and enhanced peptide (e.g. NELL1) binding by incorporation of ethyl methacrylate and N-acryloxysuccinimide; or alkyl methacrylates such as butylmethacrylate, hexylmethacrylate and dodecylmethacrylate (PCT Int. Appl. WO/2001070288; U.S. Pat. No. 5,124,151, the teachings of which are incorporated herein by reference).

In one embodiment, where the carrier may have a surface that is decorated and/or immobilized with cell adhesion molecules, adhesion peptides, and adhesion peptide analogs which may promote cell-matrix attachment via receptor mediated mechanisms, and/or molecular moieties which may promote adhesion via non-receptor mediated mechanisms binding such as, but not limited to polycationic polyamino-acid-peptides (e.g. poly-lysine), polyanionic polyamino-acid-peptides, Mefp-class adhesive molecules and other DOPA-rich peptides (e.g. poly-lysine-DOPA), polysaccharides, and proteoglycans. See for example, PCT Int. Appl. WO/2004005421; WO/2003008376; WO/9734016, the teachings of which are incorporated herein by reference.

In one embodiment, the carrier may include/is comprised of sequestering agents such as, but not limited to, collagen, gelatin, hyaluronic acid, alginate, poly(ethylene glycol), alkylcellulose (including hydroxyalkylcellulose), including methylcellulose, ethylcellulose, hydroxyethylcellulose, hydroxypropylcellulose, hydroxypropyl-methylcellulose, and carboxymethylcellulose, blood, fibrin, polyoxyethylene oxide, calcium sulfate hemihydrate, apatites, carboxyvinyl polymer, and poly(vinyl alcohol). See for example, U.S. Pat. No. 6,620,406, herein incorporated by reference.

In one embodiment, the carrier may include surfactants to promote NELL1 or NELL2 stability and/or distribution within the carrier materials such as, but not limited to polyoxyester (e.g. polysorbate 80, polysorbate 20 or Pluronic F-68).

In one embodiment, the carrier may include buffering agents such as, but not limited to glycine, glutamic acid hydrochloride, sodium chloride, guanidine, heparin, glutamic acid hydrochloride, acetic acid, succinic acid, polysorbate, dextran sulfate, sucrose, and amino acids. See for example, U.S. Pat. No. 5,385,887, herein incorporated by reference. In one embodiment, the carrier may include a combination of materials such as those listed above.

By way of example, the carrier may be a PLGA/collagen carrier membrane. The membrane may be soaked in a solution of an agent including for example, NELL1 peptide, and optionally a second agent described herein.

In one embodiment, an implant for use in the human body may include a substrate that includes one or more agents described above, including for example NELL1 peptide, and optionally a second agent described herein in an anti-oesteoinflammatory effective amount.

In one embodiment, an implant for use in the human body may include a substrate having a surface that includes an agent such as NELL1 peptide, and optionally a second agent described herein in an anti-oesteoinflammatory effective amount.

In one example, a composition according to this invention may be contained within a time release tablet.

An agent such as a NELL-1 protein or peptide, and optionally a second agent described herein peptide may be combined with an acceptable carrier to form a pharmacological composition. Acceptable carriers can contain a physiologically acceptable compound that acts, for example, to stabilize the composition or to increase or decrease the absorption of the agent. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the antimitotic agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of a carrier, including a physiologically acceptable compound depends, for example, on the route of administration.

The compositions can be administered in a variety of unit dosage forms depending upon the method of administration. For example, unit dosage forms suitable may include powder, tablets, pills, capsules.

The compositions of this invention may comprise a solution of an agent such as a NELL-1 protein or peptide, and optionally a second agent described herein peptide dissolved in a pharmaceutically acceptable carrier, such as an aqueous carrier for water-soluble peptides. A variety of carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The concentration of an agent such as a NELL-1 protein or peptide, and optionally a second agent described herein in these formulations can vary widely, and will be selected primarily based on fluid volumes, viscosities, body weight and the like in accordance with the particular mode of administration selected and the patient's needs.

### Methods of Fabrication

In one aspect of the present invention, it is provided a method of forming a composition, comprising:

providing an effective amount of an osteoinflammation preventive agent for cartilage arthritis, the osteoinflammation preventive agent comprising NELL-1 protein or peptide optionally in combination with a pro-chondrogenic agent, and

forming the composition.

In some embodiments of the invention method, optionally in combination with any of the various embodiments disclosed herein, the osteoinflammation preventive agent further comprises a non-steroidal anti-inflammatory drug (NSAID).

In some embodiments of the invention method, optionally in combination with any of the various embodiments disclosed herein, the NELL-1 protein or peptide is in an amount effective for anti-osteoinflammation.

In some embodiments of the invention method, optionally in combination with any of the various embodiments disclosed herein, the composition comprises the pro-chondrogenic agent, wherein the pro-chondrogenic agent comprises the NELL-1 protein or peptide.

In some embodiments of the invention method, optionally in combination with any of the various embodiments disclosed herein, the articular cartilage arthritis is osteoarthritis.

In some embodiments of the invention method, optionally in combination with any of the various embodiments disclosed herein, the composition is in a formulation for injection.

In some embodiments of the invention method, optionally in combination with any of the various embodiments disclosed herein, the composition is in a formulation for transdermal delivery system.

In some embodiments of the invention method, optionally in combination with any of the various embodiments disclosed herein, the subject is a human being.

### Methods of Use

In one aspect of the present invention, it is provided a method of treating cartilage arthritis in a subject in need thereof, comprising:

administering an effective amount of a pro-chondrogenic agent to the subject; and

administering an effective amount of an osteoinflammation preventive agent to the subject,

thereby ameliorating or treating at least an osteoinflammatory cartilage condition in the subject,

wherein the pro-chondrogenic agent promotes cartilage regeneration in the subject, and

wherein the osteoinflammation preventive agent reduces or decreases an osteoinflammation condition in the subject.

In some embodiments of the invention method, optionally in combination with any of the various embodiments disclosed herein, the osteoinflammation preventive agent comprises NELL-1 protein or peptide.

In some embodiments of the invention method, optionally in combination with any of the various embodiments disclosed herein, the composition comprises the pro-chondrogenic agent, wherein the pro-chondrogenic agent comprises the NELL-1 protein or peptide.

In some embodiments of the invention method, optionally in combination with any of the various embodiments disclosed herein, the NELL-1 is in a formulation for injection at a site of cartilage arthritis of the subject.

In some embodiments of the invention method, optionally in combination with any of the various embodiments disclosed herein, the NELL-1 is administered to the subject by injection at least one time per day over a treatment course.

In some embodiments of the invention method, optionally in combination with any of the various embodiments disclosed herein, the osteoinflammatory cartilage arthritis is osteoarthritis (OA).

In some embodiments of the invention method, optionally in combination with any of the various embodiments disclosed herein, the NELL-1 is administered to the subject by injection at least one time per day over a treatment course from 1 day to about 365 days.

In some embodiments of the invention method, optionally in combination with any of the various embodiments disclosed herein, the NELL-1 protein or peptide is exogenic NELL-1 protein or peptide.

In some embodiments of the invention method, optionally in combination with any of the various embodiments disclosed herein, the NELL-1 protein or peptide is administered by a recombinantly engineered cell expressing the NELL-1 protein or peptide.

In some embodiments of the invention method, optionally in combination with any of the various embodiments disclosed herein, the osteoinflammation preventive agent further comprises a non-steroidal anti-inflammatory drug (NSAID).

In some embodiments of the invention method, optionally in combination with any of the various embodiments disclosed herein, the NELL-1 protein or peptide is provided by a transdermal delivery system.

In some embodiments of the invention method, optionally in combination with any of the various embodiments disclosed herein, the subject is a human being.

### EXAMPLES

The embodiments of the present invention will be illustrated by the following set forth examples. All parameters and data are not to be construed to unduly limit the scope of the embodiments of the invention.

### Example 1. Studies on NELL-1 on Osteoarthritis

In human arthritis articular cartilage, inflammation is associated with reduced neural EGFL like 1 (NELL-1) expression. As a loss-of-function animal model, Nell-1-haploinsufficient (Nell-1^{+/6R}) mice have accelerated and aggravated osteoarthritis progression accompanied with elevated inflammatory markers. NELL-1 strongly suppressed the expression of inflammatory cytokines as well as their downstream catabolic enzymes that are responsible for cartilage extracellular matrix degradation in mouse and human articular cartilage chondrocytes. Moreover, NELL-1 administration significantly reduced the inflammatory reaction and articular cartilage damages in vivo. Excitingly, the inflammatory induced heavy ‘lame-walking’ and ‘fear-to-walk’ symptoms observed in Nell-1^{+/6R} mice were significantly recovered by the NELL-1 application.

By examining human arthritis cartilage samples as well as animal models simulating arthritis, we demonstrate the emerging role of NELL-1 in arthritis pathogenesis and identify NELL-1 as a promising anti-osteoinflammation agent for preventing and suppressing arthritis-related cartilage damages. Thus this invention provides the fundamental of clinical application of NELL-1 as a disease-modifying anti-arthritis agent. Additionally, by revealing the essential function of NELL-1 as a promising anti-inflammation and pro-chondrogenesis agent for preventing and suppressing arthritis-related cartilage damages, this invention provides the fundamental of clinical application of NELL-1 as a disease-modifying anti-arthritis agent.

### Example 2. Studies on NELL-1 as Pro-Chondrogenic, Anti-Inflammatory Dual-Functional Disease-Modifying Osteoarthritis Drug

### Summary

Arthritis, an inflammatory condition that causes pain and cartilage destruction in joints, affects over 54.4 million people in the US alone. Here, for the first time, we demonstrated the emerging role of neural EGFL like 1 (NELL-1) in arthritis pathogenesis by showing that Nell-1-haploinsufficient (Nell-1^{+/6R}) mice had accelerated and aggravated osteoarthritis (OA) progression with elevated inflammatory markers in both spontaneous primary OA and chemical-induced secondary OA models. In the chemical-induced OA model, intra-articular injection of interleukin (IL)1β induced more severe inflammation and cartilage degradation in the knee joints of Nell-1^{+/6R} mice than in wildtype animals. Mechanistically, in addition to its pro-chondrogenic potency, NELL-1 also effectively suppressed the expression of inflammatory cytokines and their downstream cartilage catabolic enzymes by upregulating runt-related transcription factor (RUNX)1 in mouse and human articular cartilage chondrocytes. Notably, NELL-1 significantly reduced IL1β-stimulated inflammation and damage to articular cartilage in vivo. In particular, NELL-1 administration markedly reduced the symptoms of antalgic gait observed in IL1β-challenged Nell-1^{+/6R} mice. Therefore, NELL-1 is a promising pro-chondrogenic, anti-inflammatory dual-functional disease-modifying osteoarthritis drug (DMOAD) candidate for preventing and suppressing arthritis-related cartilage damage.

### Introduction

In order to combat the cartilage destruction seen in OA [4], the recent search for new OA therapeutics is shifting from synthetic chemicals to biological molecules, with a specific focus on pro-chondrogenic growth factors [7,15-20]. For instance, neural EGFL-like 1 (NELL-1) is a novel pro-chondrogenic molecule that enhances the proliferation, chondrogenic differentiation, and maturation of chondrogenic-committed cells and their progenitors in vitro [21,22]. As an extra-cellular matrix (ECM) molecule expressed in articular cartilage [21], NELL-1 alone is sufficient to promote cartilage regeneration without osteophyte formation in rabbit knee critical-sized, full-thickness sub-chondral defects [23]. Moreover, our recent studies identified a novel signaling cascade of NELL-1→nuclear factor of activated T-cells (NFATc)1→runt-related transcription factor (RUNX)3→Indian hedgehog (IHH) in articular chondrocytes, which is essential for NELL-l's pro-chondrogenic bioactivities [22,24,25]. Inspired by the genome-wide association study (GWAS) that associated single nucleotide poly-morphisms (SNPs) within the NELL-1 gene with ankylosing spondylitis and psoriatic arthritis [26-28], the current study is intended to determine the role of NELL-1 in the pathogenesis of OA and its potential therapeutic benefits.

### 2. Materials and Methods

### 2.1. In Silico Prediction

In vitro half-life (t_{1/2}) and the time required for reduction to 10% of the original mature human NELL-1 protein content (t₉₀) in mammalian cells were predicted by the online server ProtParam (https://web. expasy.org/protparam/). The NELL-1 t_{1/2} is approximately 1.1 h and the t₉₀ is less than 6 h in mammalian cells in vitro. These predictions were further confirmed by ProtLifePred (http://protein-n-end-rule. leadhoster.com/).

Genomatix Software v3.10 (Genomatix AG, Munich, Germany) was used to predict the potential binding motifs of RUNX1 and NFATc1 in chondrocytes/cartilage. The sites were computationally projected with predefined transcriptional factor binding modules [29].

### 2.2. Human Arthritic Cartilage

Human arthritic cartilage samples were obtained from patients of both sexes between the ages of 32 and 92 undergoing knee arthroplasty with an institutional review board (RB) exemption since no donor identities were provided for these samples. These pre-fixed samples were used for histological and immunobiological analyses only. Meanwhile, primary adult human articular chondrocytes (hARCs) isolated from normal/healthy (NM), OA, and RA donors were purchased from Cell Application Inc. (San Diego, Calif., US) and cultured according to the manufacturer's instructions for in vitro investigations.

### 2.3. Animal Maintenance

All the experiments on live mice were performed under an in-stitutionally approved protocol provided by the Chancellor's Animal Research Committee at UCLA (protocol numbers: 2014-041 and 2013-013). Due to N-ethyl-N-nitrosourea (ENU)-induced homozygous Nell-1-deficient (Nell-1^{6R/6R}) mice having a severely reduced expression of Nell-1 that results in neonatal death [30], Nell-1-haploinsufficient (Nell-1^{+/6R}) mice (a well-established loss-of-function model [22,24,25]) were examined in the current investigation. Mice were bred and maintained as previously described [22,24,25], and their genotypes were determined by polymerase chain reaction (PCR). Genetic knockdown of Nell-1 was also confirmed by immunofluorescence (IF) staining in the tibia cartilage of newborn Nell-1^{+/6R} mouse knees (FIG. 9).

### 2.4. Primary Osteoarthritis Model

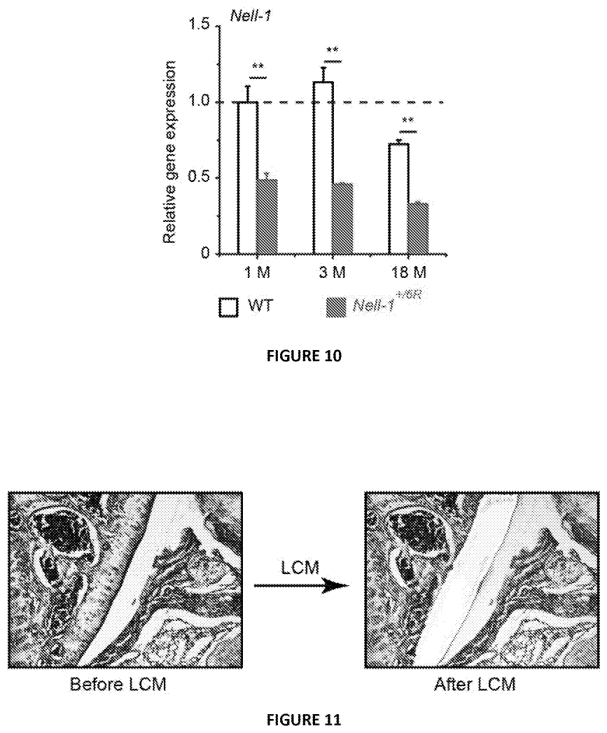

Slowly progressing OA in animals, such as in mice, closely simulates the natural progression of human primary OA [31]. Wild-type (WT) and Nell-1^{+/6R} mice at 1 month old (a prepubescent stage for mice that developmentally approximates 12.5 years of age in humans [32,33]), 3-months-old (a young mature adult stage for mice that developmentally approximates 20 years of age in humans [33]), and 18-months-old (a senescent stage for mice that developmentally approximates >50 years of age in humans [33]) were used to understand Nell-i's activities in the pathogenesis of OA. Since the prevalence of OA in human is significantly higher in women than men [4], female mice were chosen for this proof-of-concept study. Special focus was directed to the medial tibial plateau area, which is one of the most critical loadbearing areas in the body [34]. The mice were euthanized with an overdose of phenobarbital (Piramal Healthcare, Maharashtra, India), and their right hind limbs were harvested for histological and IF staining. Expression levels of Nell-1 in the medial tibial plateau cartilage were monitored using laser-capture microdissection (LCM)-coupled quantitative real-time PCR (FIG. 10) as described below.

### 2.5. Secondary Osteoarthritis Model

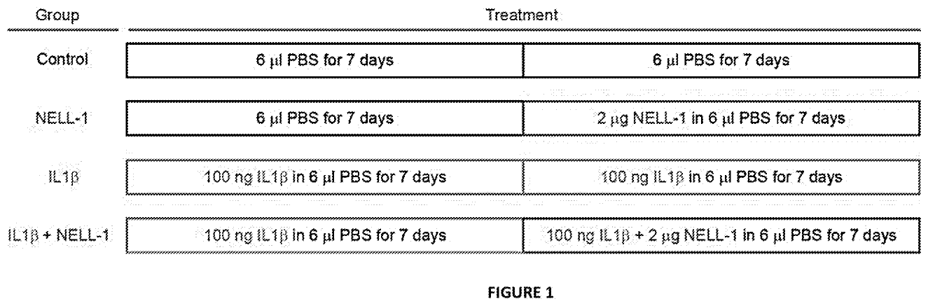

An imbalance in chondrocyte functions, which can be induced by outside stimuli such as the presence of inflammatory cytokines, leads to the progression of degenerative conditions like OA. In particular, the local elevation of IL10 in rodent knee joints has been shown to induce OA-like symptoms [35-37]. Thus, a well-documented modified direct mouse intra-articular IL1(3 injection model [38] was used to simulate secondary OA-like damage in vivo [31]. Briefly, under isoflurane anesthesia (5% for anesthesia induction and 2% for maintenance), a Hamilton syringe with a 29-gauge needle was inserted through the patellar ligament into the joint space of the right knee of 2.5-month-old female WT or Nell-1^{+/6R} mice. Since the in vivo elimination t_{1/2} of NELL-1 is 5.5 h [39], the intra-articular injection was performed twice daily by the same surgeon to avoid variation in technique. For each genotype, animals were randomly assigned to the four treatment groups (6 mice per group; FIG. 1) before the first injection: ‘Control’ group: 6 μl phosphate-buffered saline (PBS) per injection for 14 days; ‘NELL-1’ group: 6 μl PBS per injection for 7 days followed by 2 pg recombinant human NELL-1 (Aragen Bioscience, Inc., Morgan Hill, Calif., US) in 6 μl PBS per injection for another 7 days; ‘IL1β’ group: 100 ng recombinant human IL1β (PeproTech, Inc., Rocky Hill, N.J., US) in 6 μl PBS per injection for 14 days; and ‘IL1β+NELL-1’ group: 100 ng IL1β in 6 μl PBS per injection for 7 days to trigger the inflammation, while 100 ng IL1β+2 μg NELL-1 in 6 μl PBS per injection was administered for an additional 7 days. It is worth noting that, in the ‘IL1(3+NELL-1’ group, IL1β was injected along with NELL-1 in the second 7 day injection period to avoid the influence of spontaneous cartilage recovery after withholding IL1β, which was previously observed [35,40,41], and to more accurately mimic the pathological OA condition in which inflammatory stimulation is persistent. For gait analyses, videos were captured on day 7 and 14 and evaluated independently by three experienced physicians in a blinded fashion before the animals were sacrificed for histological analysis (data not shown). An established gait scoring system, which was previously used in an inflammatory monoarthritic mouse model [42], was adapted to examine impacts on animal behavior (Table 2) as recommended [43].

TABLE 2
Gait impairment criteria (adapted from [42])

| Score | Phenomena |
|---|---|
| 0 | No visible impairment of gait or stance. Foot firmly placed flat on the surface with |
| | normal spread of toes. |
| 1 | Moderate impairment of stance. Foot placed on the ground but with toes tightly |
| | contracted together. |
| 2 | Severe impairment of gait and stance. Foot either entirely elevated from the ground |
| | or only the lateral part of the foot very lightly touching the ground. Toes tightly |
| | pulled together. |

### 2.6. Histological and IF Staining

The mouse hind limb and human arthritic cartilage samples were fixed in 4% paraformaldehyde (MilliporeSigma; Burlington, Mass., US) at 4° C. for 24 h and decalcified with 19% ethylenediaminetetraacetic acid (pH 8.0; MilliporeSigma) for 21 days prior to paraffin embedding and sectioning at a thickness of 5 m. Hematoxylin and eosin (H&E) staining was performed for histological analysis, while safranin O staining was conducted with the NovaUltra™ Safranin O staining Kit (IHC World, LLC, Woodstock, Md., US) according to the manufacturer's instructions. Primary antibodies against type II collagen (II-II6B3, 1:20; Developmental Studies Hybridoma Bank, Iowa City, Iowa, US), IL1β (ab9722, 1:400; Abcam, Cambridge, Mass., US), IL6 (TA500067, 1:50; Origene, Rockville, Md., US), matrix metallopeptidase (MMP)13 (ab39012, 1:200; Abcam), bone morphogenetic protein (BMP)6 (ab155963, 1:200; Abcam), BMP7 (ab84684, 1:1000; Abcam), RUNX1 (ab35962, 1:1000, Abcam), and NELL-1 (ABP-PAB-Il648, 1:75; Allele Biotech, San Diego, Calif., US) were used for IF staining. The Vector® M.O.M.™ Immunodetection Kit (Vector Laboratories, Inc., Burlingame, Calif., US) was employed to locate mouse primary antibodies on mouse tissue. 4′,6-diamidino-2-phenylindole (DAPI; MilliporeSigma) was used for nuclear counterstaining.

### 2.7. Laser-Capture Microdissection (LCM)

For LCM, fresh-cut tissue sections at 10 μm were mounted on polyethylene naphthalate (PEN) Membrane Glass Slides (2.0 μm, MicroDissect GmbH, Herborn, Germany). To visualize the medial tibial plateau area, tissue sections were stained with Cresyl Fast Violet and completely air dried before microdissection [44]. Tibial cartilage was microdissected on a Leica LMD7000 system (Leica, Buffalo Grove, Ill., US). After microdissection, the excised region was examined microscopically (FIG. 11) and was kept on ice until RNA isolation.

### 2.8. In Vitro Mechanism of Action Investigation

To gain initial insight into the mechanism underlying NELL-1's anti-arthritic bioactivities, primary articular cartilage chondrocytes were utilized for the in vitro investigation because: (1) chondrocytes are primary contributors to articular cartilage structural support, metabolic activities, and critical maintenance functions, such as ECM secretion, within joints [45]; (2) chondrocytes are a confirmed cell source that secretes NELL-1 [21,22,24,25]; (3) local secretion of proinflammatory cytokines, including IL1β, IL6, and tumor necrosis factor (TNF)α [46-49] by articular chondrocytes can activate their autocrine loops, which are essential for the initiation and progression of arthritis [50-53]; and (4) the regulatory roles of these proinflammatory cytokines are at least partially independent from those of synovial and immune cells [53]. In particular, previous studies have confirmed that proinflammatory cytokines, including IL1β, can induce arthritis-like biological changes in articular chondrocytes in vitro [53]. Expression of proinflammatory cytokines IL1β, IL6, and TNFα, their downstream catabolic markers MMP13 and ADAM metallopeptidase with thrombospondin type 1 motif (ADAMTS)5, which are major contributors to ECM degradation during arthritis progression [54,55], the chondrocyte-secreted inflammatory maker prostaglandin-endoperoxide synthase (PTGS)2 [53], and the anabolic marker type II collagen (encoded by COL2α1) were evaluated. Based on aforementioned in silico predictions, all assessments were conducted in the 6 h post-treatment window since the t₉₀ of NELL-1 was estimated to be less than 6 h in mammalian cells.

### 2.9. Primary Mouse Articular Chondrocyte Isolation and Cultivation

Primary mouse articular cartilage chondrocytes (mARCs) were isolated and cultivated as previously described [56-58]. Briefly, small pieces of articular cartilage, located at distant sites from the synovial tissue, were removed from 3-month-old female WT or Nell-1^{+/6R} mice. These cartilage tissues were digested in 1.5 mg/ml collagenase B (MilliporeSigma) at 37° C. overnight to achieve single-cell suspensions. After rinsing with Dulbecco's Modified Eagle's Medium (DMEM), mARCs were cultured in a basal culture medium [DMEM with 10% fetal bovine serum (FBS), 100 U/ml penicillin, and 100 [μg/ml streptomycin]. The medium was changed every 3 days, and the cells were passaged at 70-90% confluence. All of these cell culture reagents were purchased from Thermo Fisher Scientific (Canoga Park, Calif., US).

### 2.10. Gene Expression Analysis

Chondrogenic-committed ATDC5 cells were obtained from the RIKEN Cell Bank (Tsukuba, Japan) and cultured in DMEM/Ham's F-12 medium (Thermo Fisher Scientific) containing 5% FBS. Passage 2 primary mARCs, isolated from 3-month-old female WT and Nell-1^{+/6R} mice, and commercially available primary hARCs were also used for gene expression analysis. Subconfluent cells were subjected to serum starvation (0.1% FBS) for 18 h and stimulated with 10 ng/ml recombinant human IL1β with or without 0.8 or 2.0 μg/ml recombinant human NELL-1.

Total RNA isolated by the RNeasy® Mini Kit (for ATDC5 cell, mARCs, or hARCs; Qiagen, Germantown, Md., US) or the RNeasy® FFPE Kit (for mouse cartilage samples obtained by LCM; Qiagen) with DNase treatment was used for reverse transcription with the iScript™ Reverse Transcription Supermix for RT-qPCR (Bio-Rad Laboratories Inc., Hercules, Calif., US). In particular, tibial cartilage samples collected from two animals were pooled together for RNA isolation to obtain enough RNA for analysis. One μl of reverse transcription product was used for real-time PCR with SsoAdvanced™ Universals Probes Supermix (Bio-Rad Laboratories Inc.) and TaqMan® primers/probe sets (Table 3; Thermo Fisher Scientific) on a QuantStudio3 system (Thermo Fisher Scientific). For each individual real-time PCR assay, three independent reserve transcription reaction products were used as templates and tested in duplicate.

TABLE 3
TaqMan ® primers/probe sets used in this study*.

| Mouse genes | | Human genes | |
|---|---|---|---|
| Gene name | Catalog number | Gene name | Catalog number |
| Gapdh | Mm99999915_g1 | GAPDH | Hs02786624_g1 |
| Adamts5 | Mm00478620_m1 | ADAMTS5 | Hs01095518_m1 |
| Col2α1 | Mm00483888_m1 | COL2α1 | Hs00264051_m1 |
| Il1β | Mm01336189_m1 | IL1β | Hs01555410_m1 |
| Il6 | Mm00446190_m1 | IL6 | Hs00174131_m1 |
| Mmp13 | Mm00439491_m1 | MMP13 | Hs00942584_m1 |
| Nfatc1 | Mm00479445_m1 | | |
| Nell-1 | Mm00616857_m1 | NELL-1 | Hs00196243_m1 |
| Ptg2 | Mm03294838_g1 | PTG2 | Hs00153133_m1 |
| Runx1 | Mm01213404_m1 | RUNX1 | Hs02558380_s1 |
| Tnfα | Mm00443258_m1 | TNF α | Hs00174128_m1 |

*All TaqMan ® primers/probe sets used in this study were purchased from Thermo Fisher Scientific.

### 2.11. Enzyme-Linked Immunosorbent Assay (ELISA)

The mouse IL-6 Platinum ELISA Kit (Cat. #BMS603-2), mouse TNF alpha Uncoated ELISA Kit (Cat. #88-7324), human IL-6 ELISA Kit (Cat. #BMS213-2), human TNF alpha Uncoated ELISA Kit (Cat. #88-7346), and human MMP-13 ELISA Kit (Cat. #EHMMP13) were purchased from Thermo Fisher Scientific, while the mouse Mmp13 ELISA Kit was purchased from MyBioSource.com (Cat. #MBS2884671; San Diego, Calif., US). Five×10⁴ cell/well in 24-well plates were treated with 1 ml medium. Cell culture medium was collected 6 h post-treatment for ELISA analyses according to the manufacturers' instructions.

### 2.12. Reduced Representation Bisulfite Sequencing (RRBS)

RRBS was conducted by the Technology Center for Genomics & Bioinformatics at UCLA. Briefly, gDNA was extracted from NM-, OA-, and RA-hARCs using the AllPrep DNA/RNA/Protein Mini Kit (Qiagen). Library preparation began by using the Nextflex® Bisulfite-Seq Library Prep Kit followed by a MspI restriction enzyme digestion (PerkinElmer, Waltham, Mass., US). First, digestion was performed, end-repair and ligation of Met-Seq adapters followed, and size selection occurred subsequently. Bisulfite conversion was performed using the EZ DNA Methylation-Gold Kit (Zymo Research, Irvine, Calif., US). The subsequent step consisted of PCR amplification for 17 cycles. This library was sequenced on a 150 bp, pair-end, HiSeq 3000 (Illumina, San Diego, Calif., US) sequencing run. Data quality checks were performed on the Illumina SAV. Demultiplexing was performed with the Illumina Bcl2fastq2 v2.17 program. Sequencing data were aligned to the GRCh37 (hg19) genome via Bismark [59]. Alignment was quantified and translated to total CpG count using Bismark's Methylation Extractor module. More than 90% of the reads were aligned to the genome (Table 4), which was in the standard range for RRBS. Differential methylation was per-formed using diffmeth and annotation was performed with identgeneloc, which are modules that are included in the DMAP package [60].

TABLE 4
Quantification of cytosine methylation.

| | | |
|---|---|---|
| NM-hARCs | OA-hARCs | RA-hARCs |

Cytosine methylation

| | | | |
|---|---|---|---|
| Total C's analyzed | 628,220,744 | 453,229,698 | 506,921,191 |
| Methylated C's in CpG context | 66,718,267 | 47,467,000 | 53,571,545 |
| Methylated C's in CHG context | 1,378,863 | 1,063,060 | 1,081,814 |
| Methylated C's in CHH context | 1,985,181 | 1,561,504 | 1,611,413 |
| Unmethylated C's in CpG context | 78,520,719 | 58,340,561 | 59,983,503 |
| Unmethylated C's in CHG context | 170,905,407 | 123,418,702 | 135,832,492 |
| Unmethylated C's in CHH context | 308,712,307 | 221,378,871 | 254,840,424 |
| Methylation percentage (CpG context) | 45.9% | 44.9% | 47.2% |
| Methylation percentage (CHG context) | 0.8% | 0.9% | 0.8% |
| Methylation percentage (CHH context) | 0.6% | 0.7% | 0.6% |

Alignment

| | | | |
|---|---|---|---|
| Unique alignment | 67.4% | 65.5% | 66.3% |
| Multiple alignment | 25.1% | 25.3% | 25.9% |
| No alignment | 7.5% | 9.1% | 7.8% |
| No genomic sequence | 0.0% | 0.0% | 0.0% |

### 2.13. RNA Interference (RNAi)

Plasmid packages harboring shRNA that targeted mouse Runx1 (G151145_0-3) and Nfatc1 (TG5010315_A-D), respectively, were purchased from OriGene (Rockville, Md., USA) and used to create stable ATDC5 knockdown cell lines with 4 μg/ml puromycin (Thermo Fisher Scientific) following the ‘Application Guide’ provided by the vendor. A scramble control vector (TR30013) provided by OriGene was also used to establish a stable control ATDC5 cell line.

### 2.14. Statistics

All statistical analyses were conducted in consultation with the UCLA Statistical Biomathematical Consulting Clinic. The sample size for each individual experiment is presented in the respective figure legends. For the gene expression and ELISA assays, one-way ANOVA and two-sample t-test analyses were performed by OriginPro 8 (Origin Lab Corp., Northampton, Mass., USA), while the Kruskal-Wallis ANOVA, Mann-Whitney U test, or paired-sample Wilcoxon test were used to analyze gait scoring. For all data presented in this manuscript, P<0.05 (*) was considered a suggestive difference, while P<0.005 (**) was recognized as a statistically significant difference based on a recent recommendation [61].

### 3. Results

Nell-1-haploinsufficiency is prone to arthritis-like pathologic changes with increased proinflammatory cytokines in mouse knee articular cartilage.

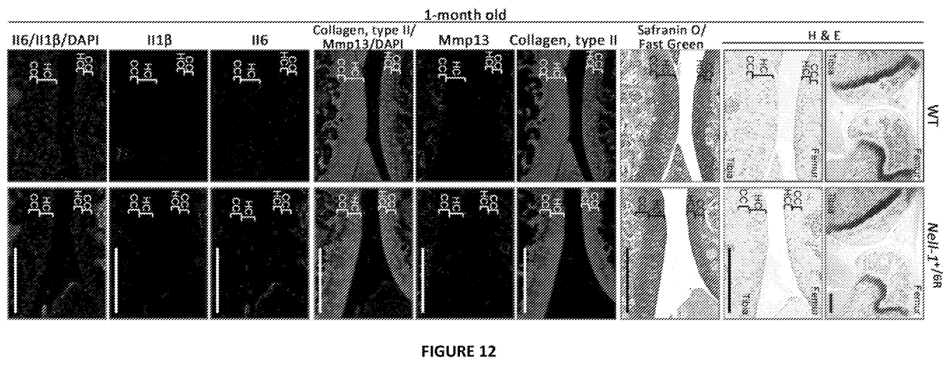

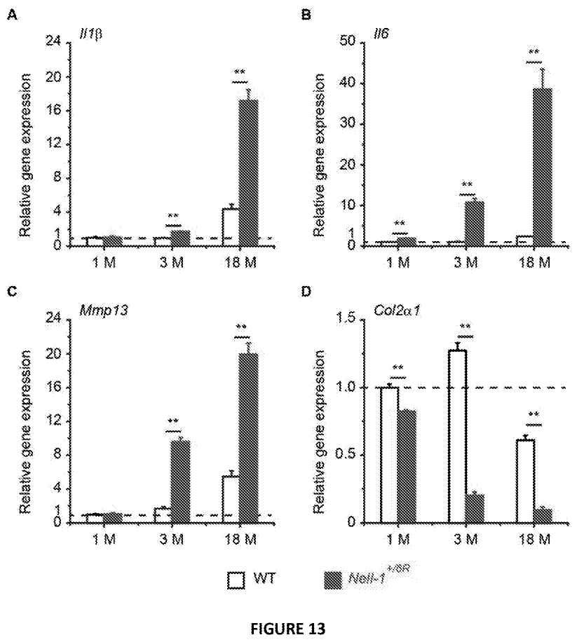

Similar to humans, mice naturally develop OA during maturation, which qualifies them as a primary OA model to study inflammation in joints [31]. At 1 month old, there were no apparent differences in cartilage degeneration or inflammation between the knees of WT and Nell-1^{+/6R} mice (FIGS. 12 and 13) except that Nell-1^{+/6R} tibial cartilage chondrocytes had slightly reduced Col2α1 expression accompanied by increased Il6 transcription (FIGS. 13B and D). Pathologically, the thicknesses of the entire articular cartilage were similar in both WT and Nell-1^{+/6R} mice. However, unlike WT mice whose uncalcified hyaline cartilage (HC) above the tidemark constituted the major portion of the cartilage, Nell-1^{+/6R} mice had cartilage in which the superficial HC and underlying calcified cartilage (CC) layer below the tidemark presented similar thicknesses, indicating that CC already expanded during the early adolescence period of Nell-1^{+/6R} mice [32].

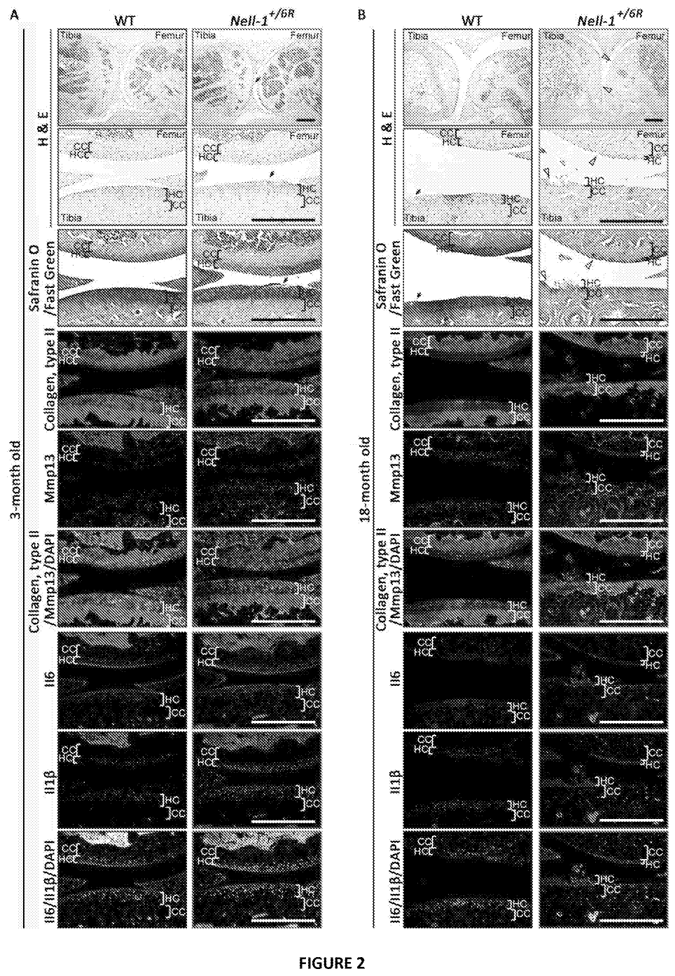

At 3 months of age, focal wear and tear of HC with early chondrocyte clustering were only observed in Nell-1^{+/6R} mice, accompanied by a lower HC/CC ratio than in age-matched WT counterparts (FIG. 2A). In comparison with WT cartilage, Nell-1^{+/6R} cartilage not only had significant type II collagen reduction and Mmp13 elevation at both the protein and RNA levels that represent key events in OA progression [54], but also had markedly pronounced 111 and Il6 expression, which indicates elevated inflammation (FIG. 2A and FIG. 13).

At 18 months of age, limited wear and tear was found in HC layer of WT cartilage with decreased type II collagen and increased Mmp13 and Il1β (FIG. 2B); a profile that exhibited great similarity to that of 3-month-old Nell-1^{+/6R} mice. In contrast, severe loss of HC [containing almost completely absent proteoglycan, negligible type II collagen, but significantly upregulated Mmp13, Il1β, and Il6 (FIG. 2B and FIG. 13)] and exposure of the underlying CC were observed in 18-month-old Nell-1^{+/6R} mouse knees. These histological and immunological changes were similar to those seen in late-stage OA of human patients [62].

Taken together, encompassing the age spectrum from juvenile, young adult, to elderly, Nell1-haploinsufficiency drastically accelerated and aggravated the arthritis-like cartilage degeneration in mice and was accompanied with significant elevation of proinflammatory cytokines.

### 3.1. Intra-Articular Injection of IL13 Induced Exaggerated OA-Like Damage in Nell-1^{+/6R} Mouse Knees

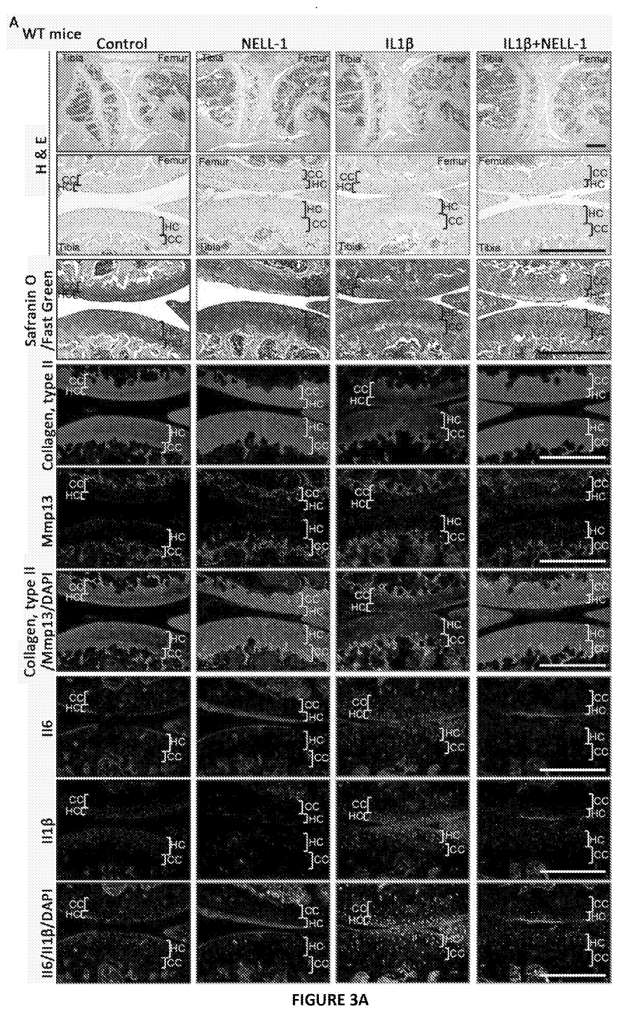

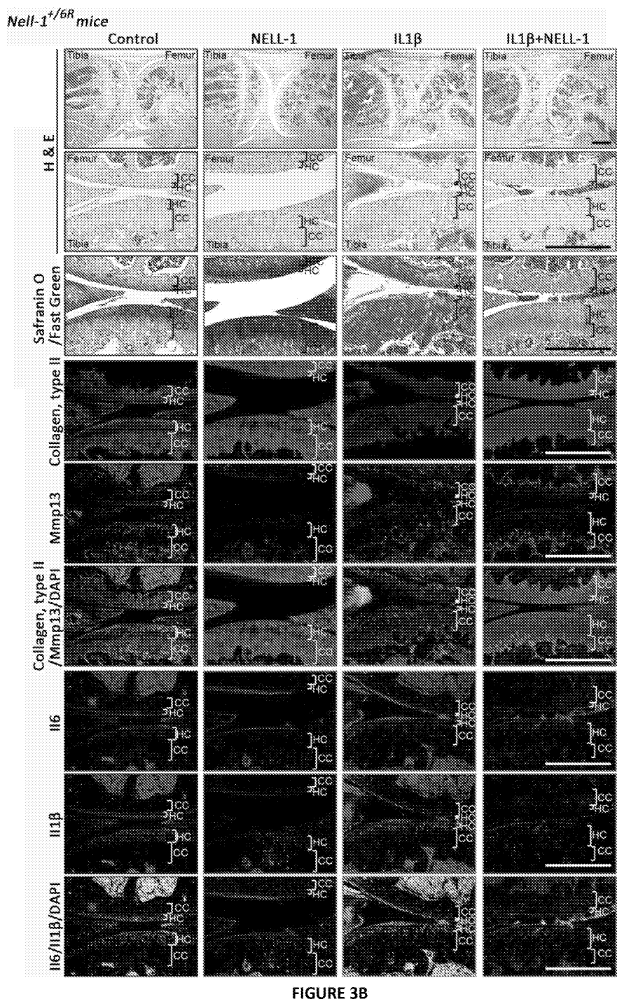

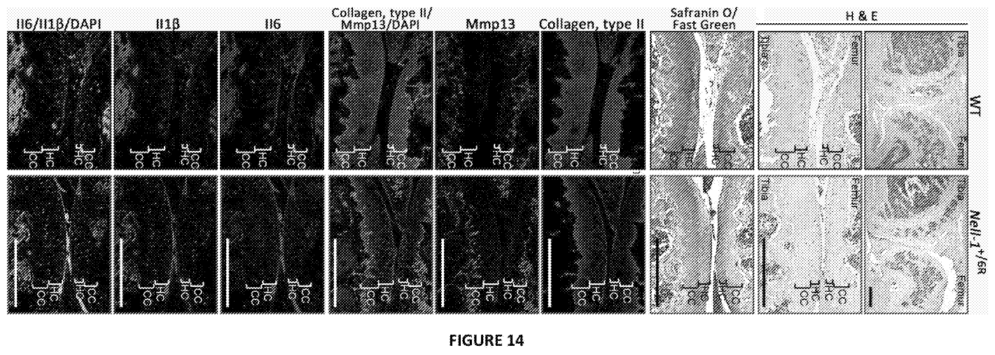

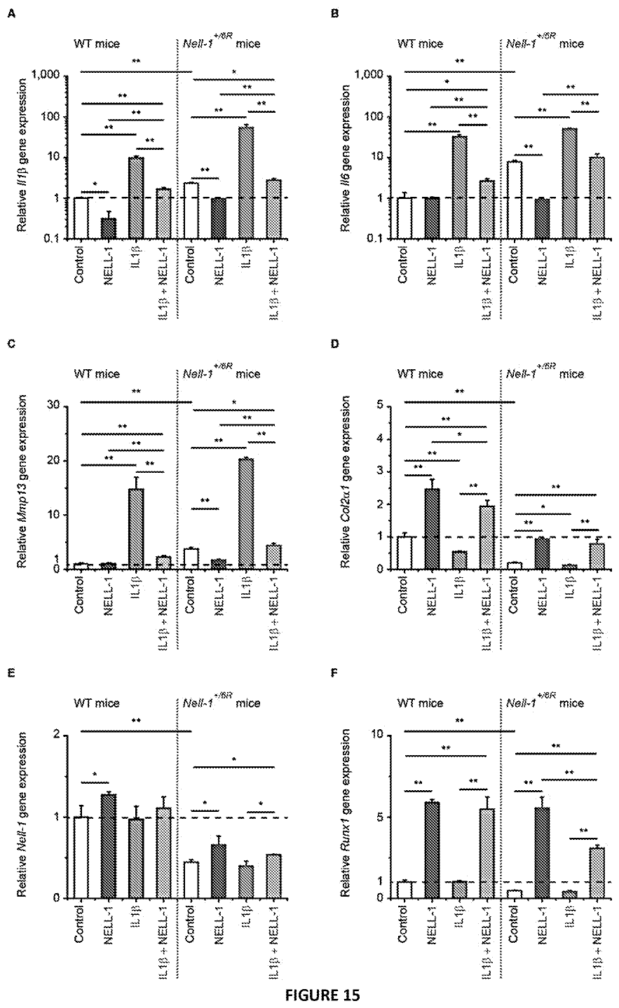

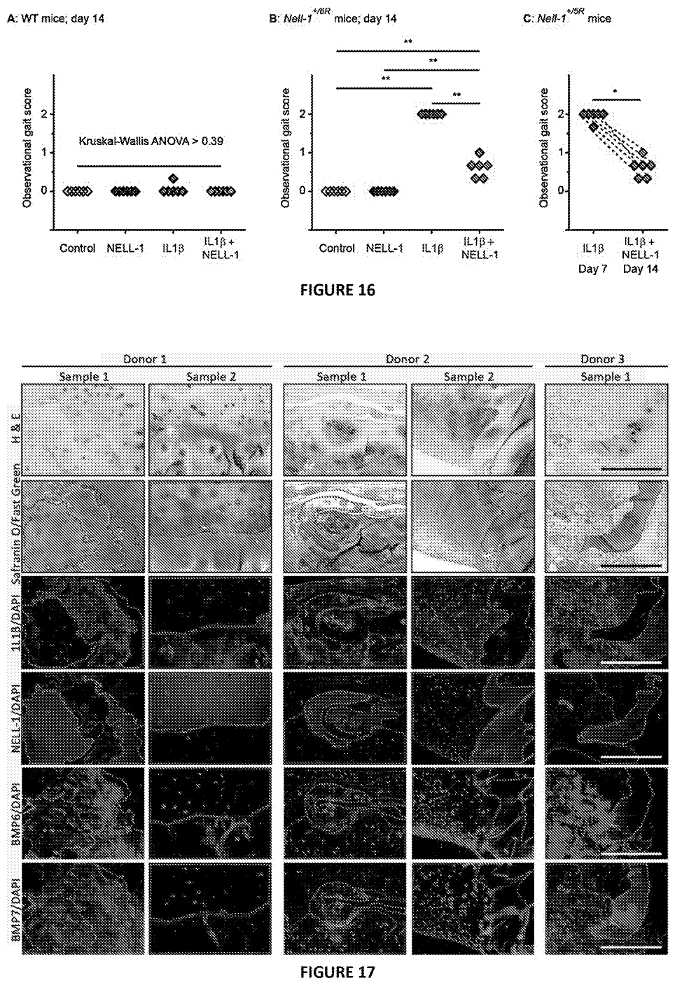

As a secondary OA model [31], 7 days of intra-articular IL1β injection (FIG. 1) was sufficient to induce proteoglycan degradation and upregulate proinflammatory cytokines Il1β and Il6 in HC of articular cartilage of 2.5-month-old WT mice (FIG. 14). Continuously challenging the joints with IL1β for 14 days led to more severe arthritis-like damage, as characterized by (1) complete abolishment of proteoglycan expression on the tibial and femoral cartilage, (2) minimal staining of type II collagen on both HC and CC, (3) increased expression of Mmp13 in HC, and (4) a significant boost in Il1β and Il6 levels in HC (FIG. 3A: ‘IL1β’, and FIG. 15A-D). However, this damage was not severe enough to significantly alter the mobility of WT mice (FIG. 16A). In comparison with age-matched WT counterparts, IL1β injection for 7 days in 2.5-month-old Nell-1^{+/6R} mouse knees resulted in a more advanced decrease of proteoglycan and type II collagen, as well as bursts of increased Mmp13, Il1β, and Il6 levels (FIG. 14). More importantly, 14 days of IL1β injection in 2.5-month old Nell-1^{+/6R} mice (3 months old at the end of treatment) replicated the drastic arthritis-like damage seen in 18-month old Nell-1^{+/6R} mice with regard to HC erosion, proteoglycan degradation, and Mmp13 expression, in addition to even more severely reduced type II collagen and increased inflammation (FIG. 3B: ‘IL1β’, and FIG. 15A-D). In congruence with the histological assessment, the symptoms of antalgic gait were observed among ‘IL1β’ group Nell-1^{+/6R} mice (FIG. 16B). Therefore, IL1β induced more severe arthritis-like damage in Nell-1^{+/6R} mouse knees.

### 3.2. NELL-1 Injection Rescued IL1β-Induced Arthritis-Like Damage in Adult Mouse Knees

To estimate the potential therapeutic benefits of NELL-1 against arthritic damage, NELL-1 was administered in the aforementioned intra-articular injection model with or without an accompanying IL1β challenge (FIG. 1). In WT mice, in comparison with a PBS vehicle control (FIG. 3A: ‘Control’), NELL-1 injections alone slightly increased the amount of proteoglycan and type II collagen with less 111 staining in HC (FIG. 3A: ‘NELL-1’). In Nell-1^{+/6R} mice, NELL-1 alone upregulated proteoglycan and type II collagen deposition in knee cartilage, while simultaneously reducing the expression of Il1β and Il6 to comparable levels of those in the age-matched WT animals (FIG. 3B: ‘NELL-1’, and FIG. 15).

When NELL-1 was administered with IL1β after the initial 7 days of IL1β-challenging, the IL1β-induced arthritis-like damage was partially rescued in WT animals, as detected by the presence of safranin O staining, upregulation of type II collagen density, and observation of Il1β and Il6 signals similar to those of the ‘Control’ group (FIG. 3A: ‘IL1β+NELL-1’, and FIG. 15). Similarly, administration of exogenous NELL-1 significantly reduced the inflammatory response and damage to articular cartilage in IL1β-challenged Nell-1^{+/6R} mice (FIG. 3B: ‘IL1+NELL-1’. and FIG. 15). Importantly, the symptoms of antalgic gait in ‘IL1β+NELL-1’ treated Nell-1^{+/6R} mice were moderate, and far less severe than the symptoms observed in ‘IL1β’ group Nell-1^{+/6R} animals (FIG. 16B). Excitingly, in a second validation experiment that tracked the mobility of ‘IL1β+NELL-1’ group Nell-1+/6R animals, the antalgic gait driven by the 7-day IL1β-challenge was drastically reduced by the subsequent 7 days of exogenous NELL-1 and IL1β administration in all 6 tested Nell-1^{+/6R} mice (FIG. 16C). Collectively, our present data demonstrate that NELL-1 has possible therapeutic potential for preventing and controlling the pathogenesis of arthritis.

### 3.3. NELL-1 Significantly Reduced IL1β-Stimulated Expression of Inflammatory and Catabolic Molecules in Mouse and Human Articular Chondrocytes In Vitro

In alignment with the aforementioned mouse models in which low Nell-1 levels correlated with high inflammation and vice versa, we observed that intense IL1β staining was generally accompanied by low levels of NELL-1, but was not necessarily associated with less BMP6 or BMP7, in human arthritic articular cartilage lesions (FIG. 17). This observation encouraged us to hypothesize that, in addition to its prochondrogenic functions, NELL-1 may also directly reduce inflammation in both mouse and human arthritis.

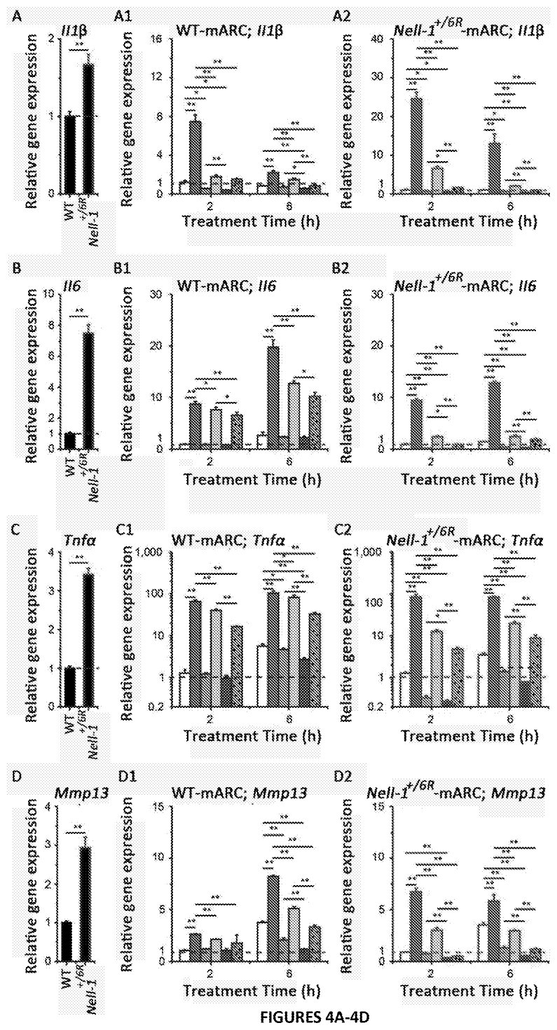

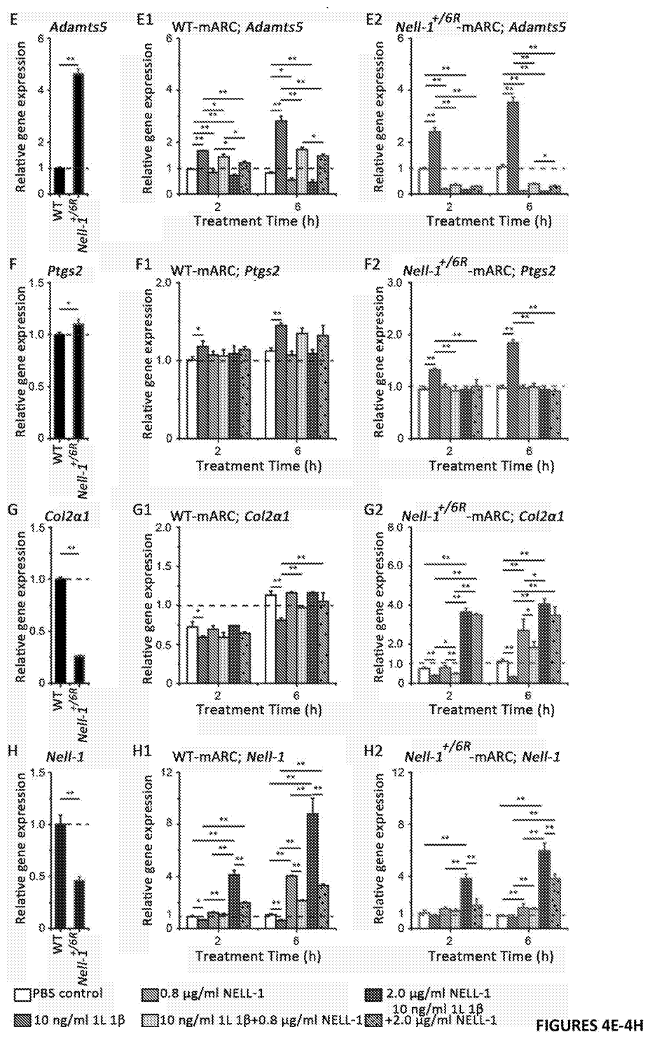

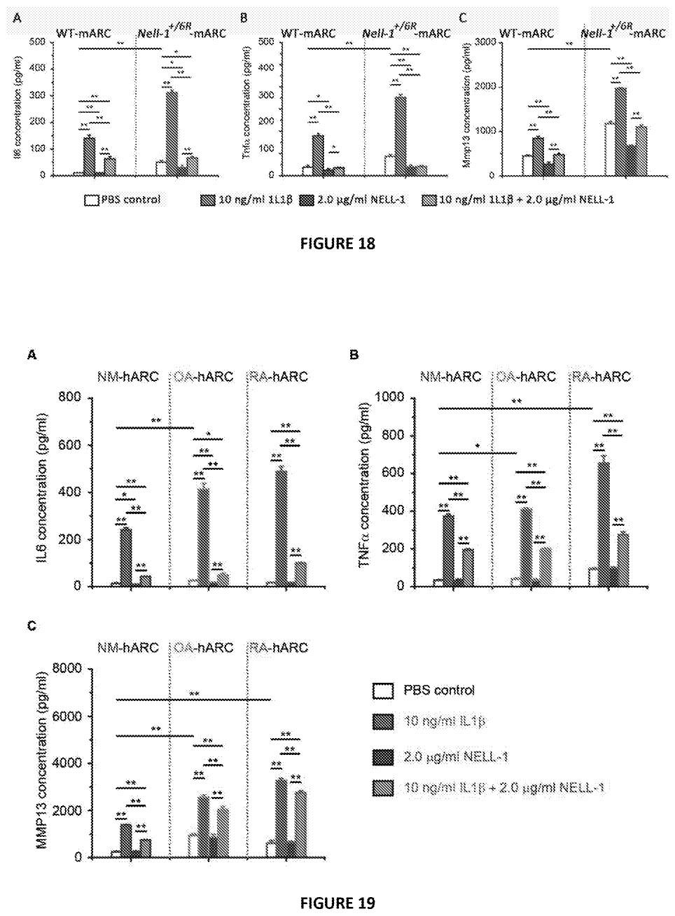

As expected, Nell-I-haploinsufficiency led to increased expression of proinflammatory and catabolic genes in primary mARCs (FIG. 4A-F). On the other hand, an apparent NELL-1 dose-dependent decline in proinflammatory and catabolic gene expression was generally observed when the mARCs were treated with NELL-1 protein alone (FIGS. 4A1-F2). Meanwhile, IL1β significantly induced the transcription of all tested proinflammatory and downstream catabolic markers in both WT- and Nell-1^{+/6R}-mARCs, which were consistently blocked by NELL-1 (FIG. 4A1-F2). The anti-inflammatory effects of NELL-1 on mARCs were further confirmed at the protein levels using ELISA (FIG. 18). In comparison with WT-mARCs, Nell-1^{+/6R}-mARCs also had lower levels of Col2α1 (which encodes the anabolic maker type II collagen; FIG. 4G), which further supports the pro-chondrogenic role of NELL-1. IL1β also markedly reduced Col2α1 expression in both WT- and Nell-1^{+/6R}-mARCs, and its effects were seemingly more pronounced in Nell-1^{+/6R}-mARCs (FIGS. 4G1-G2), while a higher dose (2 μg/ml) of NELL-1 completely eliminated the downregulation of Col2α1 caused by IL1β-stimulation or Nell-1-haploinsufficiency (FIGS. 4G1-G2). Interestingly, we also noticed that NELL-1 administration upregulated endogenous Nell-1 expression in mARCs (FIGS. 4H-H2), which is in agreement with the results from the in vivo studies (FIG. 15E). Taken together, these data reveal that NELL-1 could, at least partially, rescue mARCs from invoking chondrogenic ECM degradation and inflammation induced by endogenous Nell-1-deficiency or exogenous IL1β-stimulation.

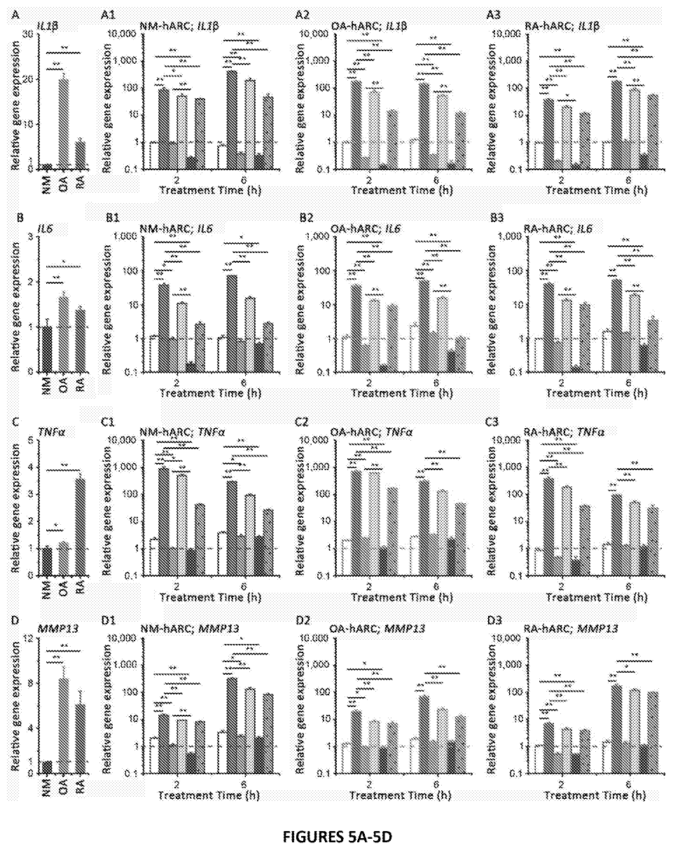

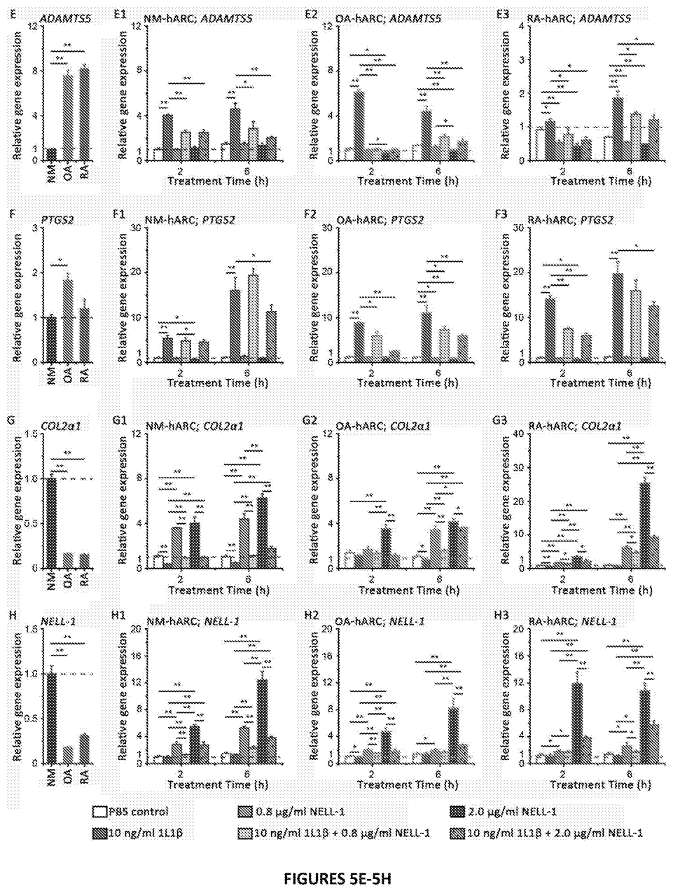

Furthermore, the aforementioned bioactivities of NELL-1 were also validated in primary hARCs isolated from NM, OA, and RA donors. In comparison with NM-hARCs, OA- and RA-hARCs had elevated levels of proinflammatory and catabolic markers, which were generally down-regulated by exogenous NELL-1 application (FIGS. 5A-F3). Due to the well-documented species-specific activity among mammalian IL1β [63,64], hARCs, in comparison with mARCs, demonstrated a greater increase in proinflammatory and catabolic gene expression in response to recombinant human IL1β administration. Further, as expected, NELL-1 actively reduced the IL1β-stimulated inflammatory responses in hARCs (FIGS. 5A1-F3 and FIG. 19). Collectively, our current data indicate that, as a pro-chondrogenic agent, NELL-1 also has a protective function against inflammation in chondrocytes, and thus, has the potential to be used as a disease-modifying osteoarthritis drug (DMOAD).

Additionally, less COL2α1 was expressed by OA- and RA-hARCs in comparison with NM-hARCs; COL2α1 transcription was reduced by IL1β and increased by NELL-1 in hARCs, which was similar to the effects of IL1β and NELL-1 seen in mARCs (FIGS. 5G-G3). Meanwhile, OA- and RA-hARCs have significantly lower levels of NELL-1 than NM-hARCs (FIG. 5H), while RA-hARCs also exhibited higher CpG methylation levels at the NELL-1 locus than NM-hARCs (Table 5), suggesting NELL-1 may also be involved in the pathogenesis of RA.

TABLE 5
CpG methylation level at NELL-1 locus of NM- and RA-hARCs.

| | | | | Number | Methylation amount | | | |
|---|---|---|---|---|---|---|---|---|
| | | | Length | of CpGs | of each sample | | | |
| | Starting | Ending | of CpG | in this | NM- | RA- | Fold | |
| | position | position | region | region | hARCs | hARCs | difference | P-value |
| 1 | 20895606 | 20895773 | 168 | 7 | 0.5980 | 0.7973 | 1.33 | 3.06E−05** |
| 2 | 20911223 | 20911372 | 150 | 7 | 0.5975 | 0.7443 | 1.20 | 0.022* |
| 3 | 20914179 | 20914330 | 152 | 5 | 0.7500 | 0.8741 | 1.17 | 0.014* |
| 4 | 21010229 | 21010428 | 200 | 10 | 0.9010 | 0.9333 | 1.04 | 0.215 |
| 5 | 21544551 | 21544687 | 137 | 7 | 0.7835 | 0.8508 | 1.09 | 0.008* |

NELL-1: GRCh37.p13, Chr 11, Location: NC_000011.9 (20691117 . . . 21597232).
*P < 0.05, a suggestive difference;
**P < 0.005, a statistically significant difference.

### 3.4. RUNX1 Mediates NELL-1's Anti-Inflammatory Activities

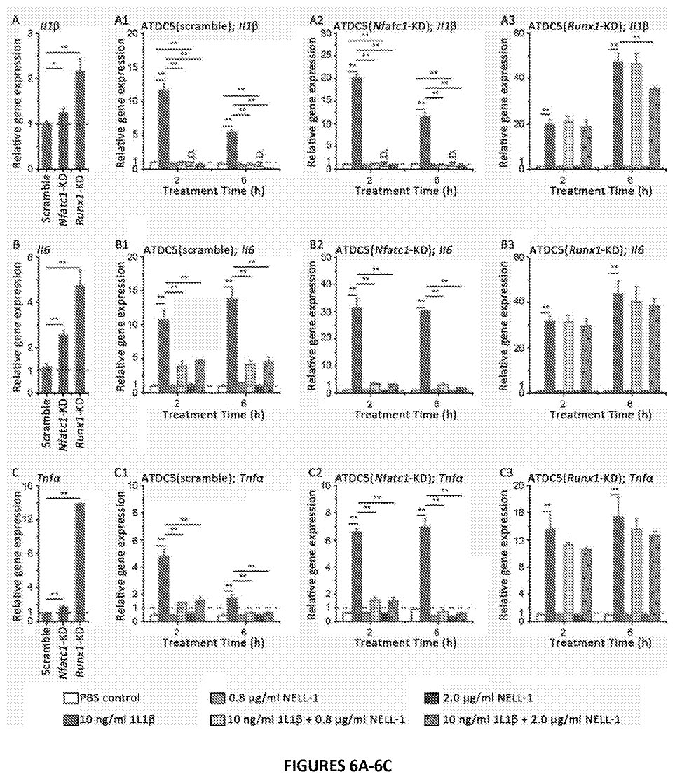

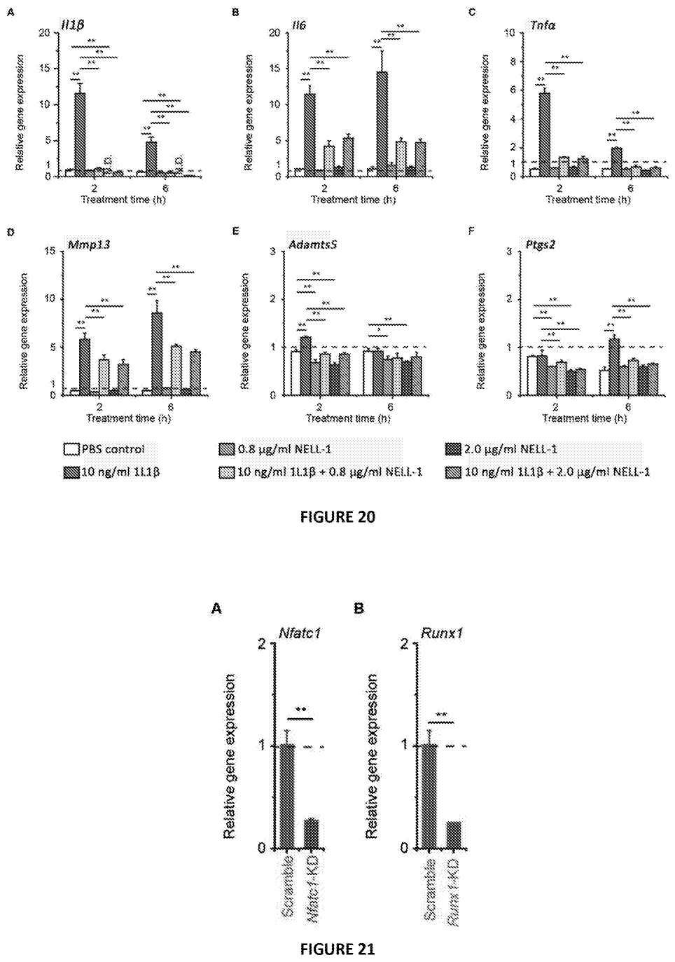

Previous studies demonstrate that NFATc1 and RUNX1 are primary genes that respond to NELL-1 in chondrocytes [25]. An in silico bioinformatic prediction indicates that both NFATc1 and RUNX1 potentially bind to promoters of IL1β and/or TNFα in human and mouse cartilage/chondrocytes (Table 6). Since the anti-inflammatory effects of NELL-1 on primary mARCs and hARCs were replicated in ATDC5 cells (FIG. 20), RNAi was used to establish stable Nfatc1- and Runx1-knockdown (KD) ATDC5 cells, respectively (FIG. 21), to examine whether RUNX1 and/or NFATc1 mediate NELL-1's anti-inflammatory activities in chondrocytes. In agreement with previous observations that both RUNX1 and NFATc1 are negative regulators of inflammation in arthritic conditions [65-68], Nfatc1- and Runx1-KD ATDC5 cells had higher endogenous levels of Il1β, Il6, and Tnfα (FIGS. 6A-C). Moreover, IL1β administration induced more pronounced elevation of these proinflammatory genes in Nfatc1- and Runx1-KD ATDC5 cells than in the scramble control plasmid transfected (scramble) ATDC5 cells (FIGS. 6A1-C3).

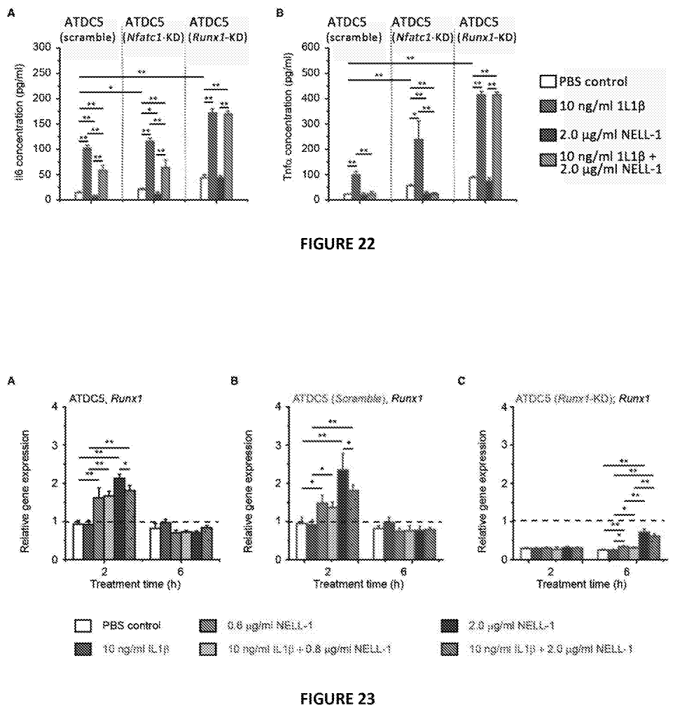

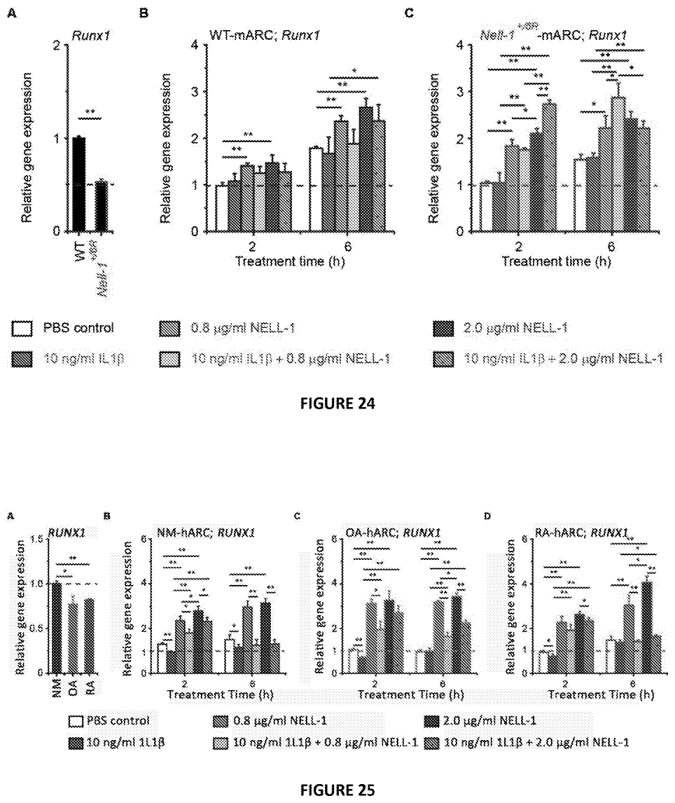

NELL-1's anti-inflammatory effects were conserved in the Nfatc1-KD ATDC5 cells (FIGS. 6A2-C2) at the same level as in the scramble ATDC5 cells (FIGS. 6A1-C1). On the contrary, Runx1-KD almost completely eliminated NELL-1's anti-inflammatory bioactivity in ATDC5 cells (FIGS. 6A3-C3 and FIG. 22). Interestingly, in comparison with non-transfected or scramble ATDC5 cells, although Runx1 upregulation was largely reduced and postponed in Runx1-KD ATDC5 cells (FIG. 23), the leaking Runx1 elevation induced by a high dose (2 μg/ml) of NELL-1 at 6 h post-treatment was still sufficient to markedly weaken the IL1β-responsive expression of Il1β in Runx1-KD ATDC5 cells (FIG. 6A3). This phenomenon further supports the hypothesis that RUNX1 is essential and adequate to render NELL-1's anti-inflammatory activity in chondrocytes. We also observed that Nell-1^{+/6R} mARCs had decreased Runx1 expression (FIG. 24A), while OA- and RA-hARCs had lower RUNX1 levels in comparison with NM-hARCs (FIG. 25A). Moreover, NELL-1 significantly upregulated Runx1/RUNX1 in all tested primary mARCs and hARCs in vitro (FIGS. 24B and C and FIGS. 25B-D). This gene expression alteration has been further confirmed at the protein level in the aforementioned intra-articular injection model in vivo: intra-articular NELL-1 administration upregulated Runx1 protein in mouse knee cartilage,

TABLE 6
In silico bioinformatics prediction of the RUNX1 and
NFATc1 binding sites on the promoters of IL1β and TNFα.

| | Tran- | | | | | | Matrix | SEQ |
|---|---|---|---|---|---|---|---|---|
| | scription | | | | | | simi- | ID |
| Species | factor | Gene* | Start | End | Strand | Sequence | larity | NO |
| Human | RUNX1 | IL1β | 816 | 830 | (−) | 5′-gggtgtggtggggtg-3′ | 0.985 | 3 |
| | | TNFα | 69 | 83 | (−) | 5′-tgctgtggtcacatc-3′ | 0.991 | 4 |
| | | | 1229 | 1243 | (−) | 5′-tgctgtggtcacatc-3′ | 0.991 | 5 |
| | | | 629 | 643 | (−) | 5′-gtctgtggtctgttt-3′ | 0.987 | 6 |
| | | | 1789 | 1803 | (−) | 5′-gtctgtggtctgttt-3′ | 0.987 | 7 |
| | NFATc1 | IL1β | 294 | 312 | (−) | 5′-ttgtccatggccacaacaa-3′ | 0.806 | 8 |
| | | | 423 | 441 | (+) | 5′-atttgcatggtgatacatt-3′ | 0.781 | 9 |
| | | | 691 | 709 | (+) | 5′-atttgcatggtgatacatt-3′ | 0.781 | 10 |
| | | | 892 | 910 | (+) | 5′-atttgcatggtgatacatt-3′ | 0.781 | 11 |
| | | TNFα | 2311 | 2329 | (−) | 5′-ctttccaggggagagaggg-3′ | 0.836 | 12 |
| | | | 286 | 304 | (+) | 5′-agttccttggaagccaaga-3′ | 0.824 | 13 |
| | | | 1446 | 1464 | (+) | 5′-agttccttggaagccaaga-3′ | 0.824 | 14 |
| | | | 2316 | 2334 | (+) | 5′-tctcccctggaaaggacac-3′ | 0.819 | 15 |
| | | | 281 | 299 | (−) | 5′-gcttccaaggaactctggg-3′ | 0.803 | 16 |
| | | | 1441 | 1459 | (−) | 5′-gcttccaaggaactctggg-3′ | 0.803 | 17 |
| Mouse | Runx1 | IL1β | 87 | 101 | (+) | 5′-gattgtggttaagaa-3′ | 0.936 | 18 |
| | Nfatc1 | IL1β | 334 | 352 | (+) | 5′-tttcccgtggaccttccag-3′ | 0.781 | 19 |
| | | TNFα | 344 | 362 | (−) | 5′-ttttccacggagcctctgc-3′ | 0.819 | 20 |
| | | | 349 | 367 | (+) | 5′-ggctccgtggaaaactcac-3′ | 0.790 | 21 |

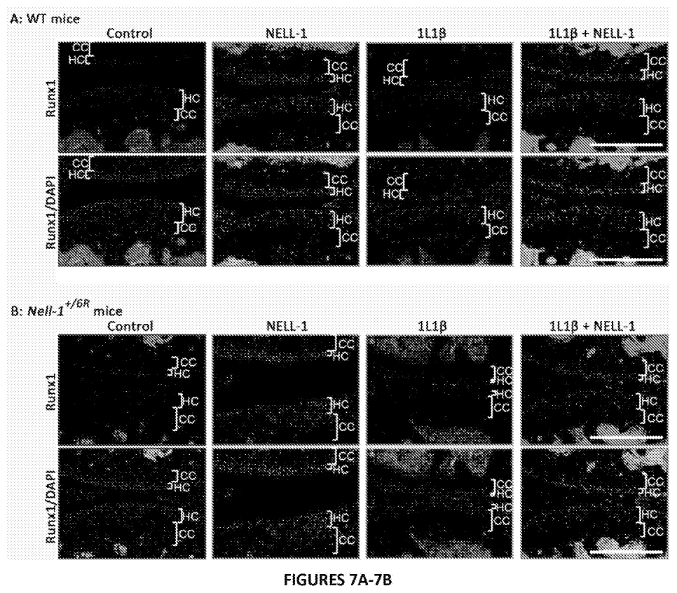

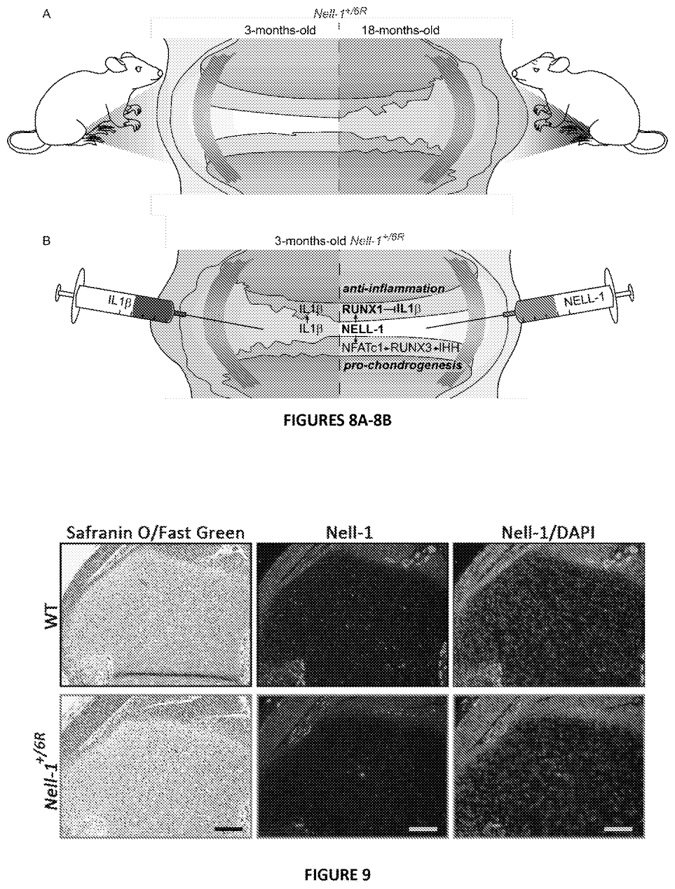

*Prediction of the binding site on the IL6/Il6 promoter was not available. which was not altered by the absence or presence of IL1β-stimulation or by Nell-1-haploinsufficiency alone (FIG. 7 and FIG. 15F). Taken together, these data suggest that RUNX1, instead of NFATc1, is a key downstream mediator of NELL-1's anti-inflammatory bioactivity in chondrocytes (FIG. 8).

### 4. Discussion

An ideal OA-combating agent that has the ability to safely reduce inflammation and promote cartilage regeneration has long been desired. The traditional use of analgesia is insufficient for curative treatment since it does not reduce inflammation and cartilage damage [5-7]; multiple adverse side-effects in the musculoskeletal, cardiovascular, and gastrointestinal systems [8,9] challenge the use of glucocorticoids as safe arthritis treatments, and NSAIDs do not effectively control arthritis progression [6]. Even more disappointing, the efficacy of DMARDs that postpone RA progression by slowing or suppressing inflammation has not been replicated in OA clinical trials via systemic or local administration [11-13]. This likely occurs because these therapeutics do not directly manage cartilage destruction—the primary cause of OA [4,5]. At this time, the prospect of using well-known pro-chondrogenic growth factors as treatments for arthritis does not appear to be optimistic either. For instance, administration of BMP7 can downregulate multiple cartilage catabolic molecules in OA-damaged tissue, but it does not notably alter proinflammatory cytokine expression [18]. Intra-articular injection of transforming growth factor (TGF)β even appears to further elevate inflammatory infiltration in treated joints [15,16], while BMP6 can induce the production of proinflammatory cytokines, such as TNFα [19], from macrophages—a major cell type responsible for inflammation and destruction in OA-ridden synovium [17].

In addition to our recent studies that revealed and confirmed the important regulatory roles of NELL-1 in chondrogenic development and maturation [21-25], we also noticed a negative correlation between proinflammatory markers and NELL-1 in both mouse and human arthritic articular cartilage. Specifically, by using spontaneous primary OA and chemical-induced secondary OA models, we further demonstrated that Nell-i-deficiency could accelerate and aggravate the progression of OA. Meanwhile, we documented a correlative decrease in NELL-1 expression with higher levels of proinflammatory cytokines found in OA-hARCs than NM-hARCs. To the best of our knowledge, this is the first time the emerging role of NELL-1 in arthritis pathogenesis has been elucidated.

Furthermore, RA-hARCs exhibited higher CpG methylation at the NELL-1 locus and lower NELL-1 transcription than NM-hARCs, which is in accordance with a previous microarray investigation that detected reduced NELL-1 expression in the damaged knee cartilage of anteromedial gonarthrosis patients [69]. In addition, SNPs within the NELL-1 gene have been detected in patients diagnosed with ankylosing spondylitis and psoriatic arthritis [26-28]. These phenomena indicate that NELL-1 may also be closely connected to the progression of a broad range of arthritis conditions; however, this observation should be further verified with a large number of arthritis patients. Moreover, determining whether alterations of NELL-1's genetic, epigenomic, and transcriptional levels are consequences or causes of continuous inflammatory infiltration should be carefully delineated in the future.

With NELL-1-stimulated chondrogenic regeneration observed in articular cartilage defects in vivo, there is an expected benefit of NELL-1 when used as arthritis therapeutic [23]. For example, when healthy, CC remains relatively constant in articular cartilage since the chondrocytes within CC typically stay quiescent during adulthood [70]. In this study, we found that intra-articular NELL-1 injection led to a moderately thickened CC layer of the tibial plateau cartilage in both WT and Nell-1^{+/6R} mice. Importantly, unlike CC reactivation in OA as a result of progressive calcification of the unmineralized cartilage that reduces the thickness of HC and the entire articular cartilage [70], NELL-1-induced CC expansion was not accompanied by noticeable HC reduction. Since recent studies revealed that articular cartilage contains mesenchymal stem cells (MSCs) and/or chondroprogenitors that are most abundant in, but not limited to, the superficial zone [71], NELL-1-induced CC expansion may result from its ability to stimulate the proliferation and chondrogenic differentiation of MSCs and chondroprogenitors [21], representing a wave of chondrogenesis in adult animals. In addition to its observed pro-chondrogenic effects, NELL-1 is able to downregulate the expression of proinflammatory and catabolic molecules, and as a result, demonstrate an anti-inflammatory potency in vitro and in vivo. Importantly, our current data demonstrate the potential of NELL-1 to rescue severe cartilage damage and reduce the symptoms of antalgic gait in an IL1β-challenged animal model that simulates OA pathogenesis, which could be attributed to NELL-1's pro-chondrogenic and anti-inflammatory dual-potency (FIG. 8). Encouragingly, previous studies have not revealed any noticeable adverse effects when NELL-1 was investigated and used for treating osteoporosis, even with systemic delivery and chemical modification that dramatically prolongs its elimination t_{1/2} and distribution in the musculoskeletal system [39,72]. Therefore, from both efficacy and safety standpoints, NELL-1 shows potential as a novel and promising DMOAD candidate in response to the unmet demand for OA therapeutics [20].

Until now, NFATc1 and NFATc2 are the most studied NELL-1 downstream effectors for chondrogenesis. Both NFATc1 and NFATc2 have been found to play important roles in maintaining cartilage health and repressing spontaneous OA [67,68]. In particular, Nfatc2^{-/-} mice develop OA between 12 and 24 months of age [73,74], which is even more markedly accelerated by cartilage-specific ablation of Nfatc1 [67]. Despite this, the current understanding of the NFATc proteins' actions in arthritis is unclear due to the controversial data. For instance, inhibition of calcineurin, an NFATc activator, decreased the severity of OA [75], while blocking glycogen synthase kinase 30, an NFATc inhibitor, induced OA in mice [76]. Further, IL1β was found to induce the expression of NFATc1 in hARCs [77]. These observations differ from previously reported anti-arthritic effects of NFATc molecules [67,68,74]. By demonstrating that NFATc1 plays an essential role in mediating NELL-1's pro-chondrogenic bioactivities via activation of the IHH signaling pathway [22,24,25], but that it is not a prerequisite for NELL-1's anti-inflammatory potency (FIG. 8), our studies provide unique insight into determining the intricate roles of NFATc1 in arthritis, which may reconcile these seemly conflicting observations.

RUNX1 is another arthritis susceptibility gene [78-80] that has been targeted for DMOAD development [65,81,82]. Aini et al. demonstrated that intra-articular injection of RUNX1 mRNA resulted in upregulated anabolic gene expression accompanied by lower 111 levels in OA mouse articular cartilage [66]. Following our previous studies that identified RUNX1 as a NELL-1-responsive gene in chondrocytes [25], this study demonstrates that RUNX1 is a key negative inflammatory regulator mobilized by NELL-1 and plays an anti-inflammatory protective role in the development of OA (FIG. 8). To our knowledge, this is the first time in which a functional upstream activator of RUNX1 has been identified for its therapeutic potency in chondrocytes.

Nevertheless, the understanding of the NELL-1→RUNX1→|IL1β functional axis is incomplete. First, the signal transduction from NELL-1 to RUNX1 is largely unknown, which may be partly due to the limited knowledge of NELL-1's specific cell surface receptor(s), associated protein(s), and downstream activators. NELL-1 may provide its function through different cell surface receptor(s) or co-receptor(s) in a cell-type- and develop-stage-dependent manner [83]. Meanwhile, the detailed mechanism of RUNX1 in arthritis is not yet clear [84,85]. Moreover, we noticed that, although IL1β did not necessarily alter articular cartilage chondrocyte NELL-1 expression in the short timeframe after exposure, the presence of IL1β profoundly blocked endogenous NELL-1 upregulation that was expected in response to exogenous NELL-1 stimulation (FIGS. 4H1-H2 and 5H1-H3). Given these facts, the interactions among NELL-1, RUNX1, and IL1β are far more complicated than they initially appear. Additionally, the mechanism behind the critical anti-arthritic autoinduction-like effect of NELL-1 in vitro (FIGS. 4H1-H2 and 5H1-5H3) and in vivo (FIG. 15E) is an interesting topic for subsequent investigation. Furthermore, the effects of NELL-1 on synovial and immune cells in the vicinity should also be assessed to fully elucidate the benefits of NELL-1 in arthritis management.

There appears to be no animal model used as the gold standard for OA [86]. In the current study, a naturally occurring OA model gave the best representation of human primary OA and a chemical-induced model simulated human secondary OA to demonstrate the importance and potential therapeutical application of NELL-1 as a DMOAD. Chemical-induced OA models (such as the intra-articular IL10 injection model used in this study) are preferred for elucidating the genetic and molecular pathogenesis and identifying targets for drug therapy since they have no correlation to post-traumatic OA [31]. However, from a clinical aspect, the anti-arthritic efficacy of NELL-1 should be further confirmed in a post-traumatic OA model since both the tested models in this study do not simulate post-traumatic OA, which constitutes 12% of all symptomatic OA cases [87]. For instance, the surgical anterior (cranial) cruciate ligament transection (ACLT) model is the earliest and the most commonly used surgical model for simulating post-traumatic OA [31,86]. Meanwhile, in comparison with small animals such as mice and rats, large animals have more anatomical and biomechanical similarities to humans [31,86]. In particular, goat knees have the closest anatomical resemblance to human knees [88]. Thus, a goat ACLT model may be useful for validating NELL-1's anti-arthritic potency and provide more clinically relevant data. The occurrence of OA is significantly higher in women [4,31], which prompted the use of female animals in our current proof-in-concept study. However, the effect of gender and reproductive status should also be evaluated in future translational studies. Lastly, the repeat intra-articular injection strategy that was used in this study is clearly not the optimal administration route for clinical treatment. Further optimization with regard to the dose and treatment regimen of NELL-1 administration should be conducted before NELL-1 can be used in clinical applications. Previous studies revealed that the in vivo elimination t_{1/2} of NELL-1 is only 5.5 h [39]; therefore, developing a suitable delivery vehicle and/or chemical modifications (such as PEGylation [39]) may also be needed to protect NELL-1 from endogenous enzyme digestion and subsequently elongate its biopotency in vivo. Taken together, considering that the investigation of NELL-1 in arthritis is in its infancy, a broad-range collaboration among academic, clinical, and therapeutic researchers is essential for facilitating the bench-to-bedside translation of this potential treatment.

### 5. Conclusions

In summary, by using a loss-of-function Nell-1^{+/6R} mouse model, we demonstrated that NELL-1 has an anti-inflammatory role to protect articular cartilage from aggravated OA progression in addition to its previously exhibited pro-chondrogenic effects. Moreover, intra-articular injection of IL1β induced more severe inflammation and cartilage degradation in the knee joints of Nell-1^{+/6R} mice than in WT control animals, while administration of exogenous NELL-1, used as a gain-of-function model, significantly reduced the inflammatory response and articular cartilage damage in both WT and Nell-1^{+/6R} mouse knees. Excitingly, the heavy antalgic gait observed in IL1 O-challenged Nell-1^{+/6R} mice notably recovered after NELL-1 administration. The anti-inflammatory effects of NELL-1 were also replicated in vitro, as evidenced by strong repression of IL1 O-stimulated inflammatory markers and their downstream catabolic enzymes that are responsible for cartilage ECM degradation. By taking advantage of RNAi technology, we demonstrate that RUNX1, instead of NFATc1, mediates the anti-inflammatory activities of NELL-1 in chondrocytes. Collectively, for the first time, our current study not only demonstrates the emerging role of NELL-1 in arthritis pathogenesis but also introduces NELL-1 as a promising new-generation DMOAD for preventing and suppressing arthritis-related cartilage damage on account of its pro-chondrogenic and anti-inflammatory potency, both of which are absent in currently available OA medications. Future investigation is strongly encouraged to uncover the detailed underlying mechanism and optimize the dose, regimen, and delivery method for transferring NELL-1-based therapies into clinical practice.

### REFERENCES

[1] Arthritis. NHS of UK, Available from: https://www.nhs.uk/conditions/arthritis/.
[2] Arthritis types. CDC of US, Available from: https://www.cdc.gov/arthritis/basics/types.html.
[3] Understanding arthritis. Arthritis foundation, Available from: https://www. arthritis.org/about-arthritis/understanding-arthritis/.
[4] International ORS, Osteoarthritis: A Serious Disease, Osteoarthritis Research Society International, 2016, pp. 1-103.
[5] Appleton C T, Osteoarthritis year in review 2017: Biology, Osteoarthr. Cartil. 26 (2018) 296-303.
[6] Scott DL, Arthritis in the elderly, in: H. M. Fillit, K. Rockwood, K. Woodhouse (Eds.), Brocklehurst's Textbook of Geriatric Medicine and Gerontology, seventh ed., SAUNDERS, ELSEVIER, Philadelphia, 2010, pp. 566-576.
[7] C. Li, M. Zou, Z. Zheng, Current medication for osteoarthritis, Acta Scientific Orthopaedics 1 (2018) 09-12.
[8] C. Cooper, T. Bardin, M. L. Brandi, P. Cacoub, J. Caminis, R. Civitelli, et al., Balancing benefits and risks of glucocorticoids in rheumatic diseases and other inflammatory joint disorders: new insights from emerging data. An expert consensus paper from the European society for clinical and economic aspects of osteoporosis and osteoarthritis (ESCEO), Aging Clin. Exp. Res. 28 (2016) 1-16.
[9] J. Compston, Glucocorticoid-induced osteoporosis: an update, Endocrine 61 (2018) 7-16.
[10] G. S. Habib, W. Saliba, M. Nashashibi, Local effects of intra-articular corticosteroids, Clin. Rheumatol. 29 (2010) 347-356.
[11] G. Verbruggen, R. Wittoek, B. V. Cruyssen, D. Elewaut, Tumour necrosis factor blockade for the treatment of erosive osteoarthritis of the interphalangeal finger joints: a double blind, randomised trial on structure modification, Ann. Rheum. Dis. 71 (2012) 891-898.
[12] X. Chevalier, P. Ravaud, E. Maheu, G. Baron, A. Rialland, P. Vergnaud, et al., Adalimumab in patients with hand osteoarthritis refractory to analgesics and nsaids: a randomised, multicentre, double-blind, placebo-controlled trial, Ann. Rheum. Dis. 74 (2015) 1697-1705.
[13] X. Chevalier, P. Goupille, A. D. Beaulieu, F. X. Burch, W. G. Bensen, T. Conrozier, et al., Intraarticular injection of anakinra in osteoarthritis of the knee: a multi-center, randomized, double-blind, placebo-controlled study, Arthritis & Rheumatism-Arthritis Care & Research 61 (2009) 344-352.
[14] M. van Laar, J. V. Pergolizzi Jr., H. U. Mellinghoff, I. M. Merchante, S. Nalamachu, J. O'Brien, et al., Pain treatment in arthritis-related pain: beyond nsaids, Open Rheumatol. J. 6 (2012) 320-330.
[15] J. B. Allen, C. L. Manthey, A. R. Hand, K. Ohura, L. Ellingsworth, S. M. Wahl, Rapid onset synovial inflammation and hyperplasia induced by transforming growth factor beta, J. Exp. Med. 171 (1990) 231-247.
[16] R. A. Fava, N. J. Olsen, A. E. Postlethwaite, K. N. Broadley, J. M. Davidson, L. B. Nanney, et al., Transforming growth factor beta 1 (TGF-beta 1) induced neutrophil recruitment to synovial tissues: implications for TGF-beta-driven synovial inflammation and hyperplasia, J. Exp. Med. 173 (1991) 1121-1132.
[17] J. Bondeson, S. D. Wainwright, S. Lauder, N. Amos, C. E. Hughes, The role of synovial macrophages and macrophage-produced cytokines in driving aggrecanases, matrix metalloproteinases, and other destructive and inflammatory responses in osteoarthritis, Arthritis Res. Ther. 8 (2006) R187.
[18] N. Badlani, Y. Oshima, R. Healey, R. Coutts, D. Amiel, Use of bone morphogenic protein-7 as a treatment for osteoarthritis, Clin. Orthop. Relat. Res. 467 (2009) 3221-3229.
[19] J. H. Hong, G. T. Lee, J. H. Lee, S. J. Kwon, S. H. Park, S. J. Kim, et al., Effect of bone morphogenetic protein-6 on macrophages, Immunology 128 (2009) e442-450.
[20] C. Li, Z. Zheng, What's the future of osteoarthritis treatment, Acta Scientific Orthopaedics 1 (2018) 01-02.
[21] C. S. Li, X. Zhang, B. Peault, J. Jiang, K. Ting, C. Soo, et al., Accelerated chondrogenic differentiation of human perivascular stem cells with NELL-1, Tissue Eng. A 22 (2016) 272-285.
[22] C. Li, J. Jiang, Z. Zheng, K. S. Lee, Y. Zhou, E. Chen, et al., Neural EGFL-Like 1 is a downstream regulator of Runt-related transcription factor 2 in chondrogenic differentiation and maturation, Am. J. Pathol. 183 (2017) 963-972.
[23] R. K. Siu, J. N. Zara, Y. Hou, A. W. James, J. Kwak, X. Zhang, et al., NELL-1 promotes cartilage regeneration in an in vivo rabbit model, Tissue Eng. A 18 (2012) 252-261.
[24] C. S. Li, Z. Zheng, J. Jiang, W. L. Jiang, K. Lee, E. A. Berthiaume, et al., Neural EGFL-Like 1 regulates cartilage maturation through Runt-related transcription factor 3-mediated Indian Hedgehog signaling, Am. J. Pathol. 188 (2018) 392-403.
[25] C. S. Li, Z. Zheng, X. L. Zhang, G. Asatrian, E. Chen, R. Song, et al., Nfatc1 is a functional transcriptional factor mediating NELL-1-induced runx3 upregulation in chondrocytes, Int. J. Mol. Sci. 19 (1) (2018) 168.
[26] N. Bartolome, M. Szczypiorska, A. Sanchez, J. Sanz, X. Juanola-Roura, J. Gratacos, et al., Genetic polymorphisms inside and outside the MHC improve prediction of as radiographic severity in addition to clinical variables, Rheumatology 51 (2012) 1471-1478.
[27] P. Ho, J. Bowes, C. E. Filer, I. N. Bruce, A. Barton, Investigation of Crohn's disease and ankylosing spondylitis susceptibility loci with psoriatic arthritis, Arthritis Rheum. 58 (2008) S350-S350.
[28] Y. L. B. J. Polo, M. Szczypiorska, N. Bartolome, J. Campos, B. J. Flores-Robles, J. Sanz, et al., Clinical and genetic characteristics of ankylosing spondylitis patients with peripheral arthritis at disease onset, Clin. Exp. Rheumatol. 37 (2) (2019) 0215-0221.
[29] A. Klingenhoff, K. Frech, K. Quandt, T. Werner, Functional promoter modules can be defected by formal models independent of overall nucleoside sequence similarity, Bioinformatics 15 (1999) 180-186.
[30] J. Desai, M. E. Shannon, M. D. Johnson, D. W. Ruff, L. A. Hughes, M. K. Kerley, et al., Nell1-deficient mice have reduced expression of extracellular matrix proteins causing cranial and vertebral defects, Hum. Mol. Genet. 15 (2006) 1329-1341.
[31] E. L. Kuyinu, G. Narayanan, L. S. Nair, C. T. Laurencin, Animal models of osteoarthritis: classification, update, and measurement of outcomes, J. Orthop. Surg. Res. 11 (2016) 19.
[32] V. Brust, P. M. Schindler, L. Lewejohann, Lifetime development of behavioural phenotype in the house mouse (Mus musculus), Front. Zool. 12 (2015) S1-S17.
[33] J. Shen, A. W. James, X. Zhang, S. Pang, J. N. Zara, G. Asatrian, et al., Novel Wnt regulator NEL-like molecule-1 antagonizes adipogenesis and augments osteogenesis induced by bone morphogenetic protein 2, Am. J. Pathol. 186 (2016) 419-434.
[34] T. Fukubayashi, H. Kurosawa, The contact area and pressure distribution pattern of the knee—a study of normal and osteoarthrotic knee joints, Acta Orthop. Scand. 51 (1980) 871-879.
[35] A. A. van de Loo, W. B. van den Berg, Effects of murine recombinant interleukin 1 on synovial joints in mice: measurement of patellar cartilage metabolism and joint inflammation,
Ann. Rheum. Dis. 49 (1990) 238-245.
[36] H. M. van Beuningen, P. M. van der Kraan, O. J. Arntz, W. B. van den Berg, In vivo protection against interleukin-1-induced articular cartilage damage by transforming growth factor-beta 1: age-related differences, Ann. Rheum. Dis. 53 (1994) 593-600.
[37] K. D. Allen, S. B. Adams, B. A. Mata, M. F. Shamji, E. Gouze, L. F. Jing, et al., Gait and behavior in an IL1 beta-mediated model of rat knee arthritis and effects of an IL1 antagonist, J. Orthop. Res. 29 (2011) 694-703.
[38] A. A. J. Vandeloo, W. B. Vandenberg, Effects of murine recombinant interleukin-1 on synovial joints in mice—measurement of patellar cartilage metabolism and joint inflammation, Ann. Rheum. Dis. 49 (1990) 238-245.
[39] J. H. Kwak, Y. L. Zhang, J. Park, E. Chen, J. Shen, C. Chawan, et al.,
Pharmacokinetics and osteogenic potential of Pegylated NELL-1 in vivo after systemic administration, Biomaterials 57 (2015) 73-83.
[40] C. Lundberg, I. Asberg, M. Ionescu, A. Reiner, G. Smedegard, A. R. Poole, Changes in cartilage proteoglycan aggrecan after intra-articular injection of interleukin-1 in rabbits: studies of synovial fluid and articular cartilage, Ann. Rheum. Dis. 55 (1996) 525-534.
[41] D. P. Page Thomas, B. King, T. Stephens, J. T. Dingle, In vivo studies of cartilage regeneration after damage induced by catabolin/interleukin-1, Ann. Rheum. Dis. 50 (1991) 75-80.
[42] U. Heilborn, O. G. Berge, L. Arborelius, E. Brodin, Spontaneous nociceptive behaviour in female mice with Freund's complete adjuvant- and carrageenan-induced monoarthritis, Brain Res. 1143 (2007) 143-149.
[43] E. H. Lakes, K. D. Allen, Gait analysis methods for rodent models of arthritic disorders: reviews and recommendations, Osteoarthr. Cartil. 24 (2016) 1837-1849.
[44] P. Amini, J. Ettlin, L. Opitz, E. Clementi, A. Malbon, E. Markkanen, An optimised protocol for isolation of RNA from small sections of laser-capture microdissected FFPE tissue amenable for next-generation sequencing, BMC Mol. Biol. 18 (2017) 22.
[45] H. Akkiraju, A. Nohe, Role of chondrocytes in cartilage formation, progression of osteoarthritis and cartilage regeneration, J. Dev. Biol. 3 (2015) 177-192.
[46] P. Kumar, S. Banik, Pharmacotherapy options in rheumatoid arthritis. Clinical Medicine Insights, Arthritis Musculoskeletal Disorders 6 (2013) 35-43.
[47] M. Kapoor, J. Martel-Pelletier, D. Lajeunesse, J. P. Pelletier, H. Fahmi, Role of proinflammatory cytokines in the pathophysiology of osteoarthritis, Nat. Rev. Rheumatol. 7 (2011) 33-42.
[48] F. Berenbaum, Osteoarthritis as an inflammatory disease (osteoarthritis is not osteoarthrosis!), Osteoarthr. Cartil. 21 (2013) 16-21.
[49] J. Sokolove, C. M. Lepus, Role of inflammation in the pathogenesis of osteoarthritis: latest findings and interpretations, Therapeutic Advances in Musculoskeletal Disease 5 (2013) 77-94.
[50] B. V. Shlopov, M. L. Gumanovskaya, K. A. Hasty, Autocrine regulation of collagenase 3 (matrix metalloproteinase 13) during osteoarthritis, Arthritis Rheum. 43 (2000) 195-205.
[51] W. P. Arend, J. M. Dayer, Inhibition of the production and effects of interleukin-1 and tumor-necrosis-factor-alpha in rheumatoid-arthritis, Arthritis Rheum. 38 (1995) 151-160.
[52] M. J. Pearson, D. Herndler-Brandstetter, M. A. Tariq, T. A. Nicholson, A. M. Philp, H. L. Smith, et al., IL-6 secretion in osteoarthritis patients is mediated by chondrocyte-synovial fibroblast cross-talk and is enhanced by obesity, Sci. Rep. 7 (2017) 3451.
[53] M. B. Goldring, M. Otero, Inflammation in osteoarthritis, Curr. Opin. Rheumatol. 23 (2011) 471-478.
[54] M. N. Wang, E. R. Sampson, H. T. Jin, J. Li, Q. H. Ke, H. J. Im, et al., MMP13 is a critical target gene during the progression of osteoarthritis, Arthritis Res. Ther. 15 (2013) R5.
[55] H. Stanton, F. M. Rogerson, C. J. East, S. B. Golub, K. E. Lawlor, C. T. Meeker, et al., ADAMTS5 is the major aggrecanase in mouse cartilage in vivo and in vitro, Nature 434 (2005) 648-652.
[56] H. M. van Beuningen, R. Stoop, P. Buma, N. Takahashi, P. M. van der Kraan, W. B. van den Berg, Phenotypic differences in murine chondrocyte cell lines derived from mature articular cartilage, Osteoarthr. Cartil. 10 (2002) 977-986.
[57] Y. Y. Tan, G. Zhao, D. Wang, J. M. Wang, J. R. Tang, Z. L. Ji, A new strategy of minimally invasive surgery for cholecystolithiasis: calculi removal and gallbladder preservation, Digestive Surgury 30 (2013) 466-471.
[58] M. Gosset, F. Berenbaum, S. Thirion, C. Jacques, Primary culture and phenotyping of murine chondrocytes, Nat. Protoc. 3 (2008) 1253-1260.
[59] F. Krueger, S. R. Andrews, Bismark: a flexible aligner and methylation caller for Bisulfite-Seq applications, Bioinformatics 27 (2011) 1571-1572.
[60] P. A. Stockwell, A. Chatterjee, E. J. Rodger, I. M. Morison, DMAP: differential methylation analysis package for RRBS and WGBS data, Bioinformatics 30 (2014) 1814-1822.
[61] D. J. Benjamin, J. O. Berger, M. Johannesson, B. A. Nosek, E.-J. Wagenmakers, R. Berk, et al., Redefine statistical significance. We propose to change the default p-value threshold for statistical significance from 0.05 to 0.005 for claims of new discoveries, Nature Human Behaviour (2017) 1.
[62] J. Aubrey, Hough J. Pathology of osteoarthritis, in: R. W. Moskowitz, R. D. Altman, M. C. Hochberg, J. A. Buckwalter, V. M. Goldberg (Eds.), Osteoarthritis: Diagnosis and Medical/surgical Management, fourth ed., Wolter Kluwer/Lippincott Willams & Wilkins, Philadelphia, Pa., 2007, pp. 51-72.
[63] C. L. Liu, Y. C. Bai, D. Ganea, R. P. Hart, Species-specific activity of rat recombinant interleukin-1-beta, J. Interferon Cytokine Res. 15 (1995) 985-992.
[64] A. I. Koussounadis, D. W. Ritchie, G. J. L. Kemp, C. J. Secombes, Analysis of fish IL-1 beta and derived peptide sequences indicates conserved structures with species-specific IL-1 receptor binding: implications for pharmacological design, Curr. Pharmaceut. Des. 10 (2004) 3857-3871.
[65] F. Yano, H. Hojo, S. Ohba, A. Fukai, Y. Hosaka, T. Ikeda, et al., A novel disease-modifying osteoarthritis drug candidate targeting Runx1, Ann. Rheum. Dis. 72 (2013) 748-753.
[66] H. Aini, K. Itaka, A. Fujisawa, H. Uchida, S. Uchida, S. Fukushima, et al., Messenger RNA delivery of a cartilage-anabolic transcription factor as a disease-modifying strategy for osteoarthritis treatment, Sci. Rep. 6 (2016) 18743.
[67] M. B. Greenblatt, S. Y. Ritter, J. Wright, K. Tsang, D. Hu, L. H. Glimcher, et al., NFATc1 and NFATc 2 repress spontaneous osteoarthritis, Proc. Natl. Acad. Sci. U.S.A. 110 (2013) 19914-19919.
[68] F. Beier, NFATs are good for your cartilage!, Osteoarthr. Cartil. 22 (2014) 893-895.
[69] S. Snelling, R. Rout, R. Davidson, I. Clark, A. Carr, P. A. Hulley, et al., A gene ex-pression study of normal and damaged cartilage in anteromedial gonarthrosis, a phenotype of osteoarthritis, Osteoarthr. Cartil. 22 (2014) 334-343.
[70] T. R. Oegema, R. J. Carpenter, F. Hofmeister, R. C. Thompson, The interaction of the zone of calcified cartilage and subchondral bone in osteoarthritis, Microsc. Res. Tech. 37 (1997) 324-332.
[71] M. E. Candela, R. Yasuhara, M. Iwamoto, M. Enomoto-Iwamoto, Resident mesenchymal progenitors of articular cartilage, Matrix Biol. 39 (2014) 44-49.
[72] A. W. James, J. Shen, X. Zhang, G. Asatrian, R. Goyal, J. H. Kwak, et al., NELL-1 in the treatment of osteoporotic bone loss, Nat. Commun. 6 (2015) 7362.
[73] A. M. Ranger, L. C. Gerstenfeld, J. Wang, T. Kon, H. Bae, E. M. Gravallese, et al., The nuclear factor of activated t cells (NFAT) transcription factor NFATp (NFAT2) is a repressor of chondrogenesis, J. Exp. Med. 191 (2000) 9-22.
[74] J. Wang, B. M. Gardner, Q. Lu, M. Rodova, B. G. Woodbury, J. G. Yost, et al., 25 Transcription factor Nfatl deficiency causes osteoarthritis through dysfunction of adult articular chondrocytes, J. Pathol. 219 (2009) 163-172.
[75] S. A. Yoo, B. H. Park, H. J. Yoon, J. Y. Lee, J. H. Song, H. A. Kim, et al., Calcineurin modulates the catabolic and anabolic activity of chondrocytes and participates in the progression of experimental osteoarthritis, Arthritis Rheum. 56 (2007) 2299-2311.
[76] R. L. Miclea, M. Siebelt, L. Finos, J. J. Goeman, C. W. Lowik, W. Oostdijk, et al., Inhibition of Gsk33 in cartilage induces osteoarthritic features through activation of the canonical Wnt signaling pathway, Osteoarthr. Cartil. 19 (2011) 1363-1372.
[77] K. O. Yaykasli, T. Oohashi, S. Hirohata, O. F. Hatipoglu, K. Inagawa, K. Demircan, et al., ADAMTS9 activation by interleukin 10 via NFATc1 in OUMS-27 chondrosarcoma cells and in human chondrocytes, Mol. Cell. Biochem. 323 (2009) 69-79.
[78] M. A. Jeffries, M. Donica, L. W. Baker, M. E. Stevenson, A. C. Annan, M. B. Humphrey, et al., Genome-wide DNA methylation study identifies significant epigenomic changes in osteoarthritic cartilage, Arthritis & Rheumatology. 66 (2014) 2804-2815.
[79] Y. Wang, J. Godec, K. Ben-Aissa, K. R. Cui, K. J. Zhao, A. B. Pucsek, et al., The transcription factors T-bet and Runx are required for the ontogeny of pathogenic interferon-y-producing T helper 17 cells, Immunity 40 (2014) 355-366.
[80] S. Tokuhiro, R. Yamada, X. T. Chang, A. Suzuki, Y. Kochi, T. Sawada, et al., An intronic SNP in a RUNX1 binding site of SLC22A4, encoding an organic cation transporter, is associated with rheumatoid arthritis, Nat. Genet. 35 (2003) 341-348.
[81] K. Johnson, S. T. Zhu, M. S. Tremblay, J. N. Payette, J. N. Wang, L. C. Bouchez, et al., A stem cell-based approach to cartilage repair, Science 336 (2012) 717-721.
[82] F. J. Blanco, C. Ruiz-Romero, New targets for disease modifying osteoarthritis drugs: chondrogenesis and Runx1, Ann. Rheum. Dis. 72 (2013) 631-634.
[83] C. Li, Z. Zheng, P. Ha, X. Chen, W. Jiang, S. Sun, et al., Neurexin superfamily cell membrane receptor contactin-associated protein like-4 (Cntnap4) is involved in neural EGFL-like 1 (NELL-1)-responsive osteogenesis, J. Bone Miner. Res. 33 (2018) 1813-1825.
[84] M. E. Alarcon-Riquelme, Role of RUNX in autoimmune diseases linking rheumatoid arthritis, psoriasis and lupus, Arthritis Res. Ther. 6 (2004) 169-173.
[85] Y. H. Lee, S. C. Bae, J. H. Kim, Y. H. Seo, S. J. Choi, J. D. Ji, et al., Meta-analysis of SLC22A4 and RUNX1 polymorphisms associations with rheumatoid arthritis susceptibility, Zeitschrift Fur Rheumatologie 74 (2015) 351-358.
[86] M. H. Gregory, N. Capito, K. Kuroki, A. M. Stoker, J. L. Cook, S. L. Sherman, A review of translational animal models for knee osteoarthritis, Arthritis 2012 (2012) 764621.
[87] L. Punzi, P. Galozzi, R. Luisetto, M. Favero, R. Ramonda, F. Oliviero, et al., Post-traumatic arthritis: overview on pathogenic mechanisms and role of inflammation, RMD Open 2 (2016).
[88] B. L. Proffen, M. McElfresh, B. C. Fleming, M. M. Murray, A comparative anatomical study of the human knee and six animal species, The Knee 19 (2012) 493-499.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The teachings of the references, including patents and patent related documents, cited herein are incorporated herein in their entirety to the extent not inconsistent with the teachings herein.

We claim:

1. A method of treating a cartilage arthritis condition in a subject in need thereof, comprising
administering an effective amount of a pro-chondrogenic agent to the subject; and
administering an effective amount of an osteoinflammation preventive agent to the subject,
thereby ameliorating or treating at least an cartilage arthritis condition in the subject,
wherein the pro-chondrogenic agent promotes cartilage regeneration in the subject, and
wherein the osteoinflammation preventive agent reduces or decreases the cartilage arthritis condition in the subject.

2. The method according to claim 1, wherein the osteoinflammation preventive agent comprises NELL-1 protein or peptide.

3. The method according to claim 1, wherein the pro-chondrogenic agent comprises NELL-1 protein or peptide.

4. The method according to claim 2, wherein the NELL-1 is in a formulation for injection at a site of cartilage arthritis of the subject.

5. The method according to claim 4, wherein the NELL-1 is administered to the subject by injection at least one time per day over a treatment course.

5. The method according to claim 1, wherein the cartilage arthritis condition is osteoarthritis (OA).

6. The method according to claim 2, wherein the NELL-1 is administered to the subject by injection at least one time per day over a treatment course from 1 day to about 365 days.

7. The method according to claim 2, wherein the NELL-1 protein or peptide is exogenic NELL-1 protein or peptide.

8. The method according to claim 2, wherein the NELL-1 protein or peptide is administered by a recombinantly engineered cell expressing the NELL-1 protein or peptide.

9. The method according to claim 2, wherein the osteoinflammation preventive agent further comprises a non-steroidal anti-inflammatory drug (NSAID).

10. The method according to claim 2, wherein the NELL-1 protein or peptide is provided by a transdermal delivery system.

11. The method according to claim 1, wherein the subject is a human being.

12. A composition for cartilage arthritis in a subject, comprising an effective amount of an osteoinflammation preventive agent for cartilage arthritis, the osteoinflammation preventive agent comprising NELL-1 protein or peptide optionally in combination with a pro-chondrogenic agent.

13. The composition according to claim 12, wherein the osteoinflammation preventive agent further comprises a non-steroidal anti-inflammatory drug (NSAID).

14. The composition according to claim 13, wherein the NELL-1 protein or peptide is in an amount effective for anti-osteoinflammation.

15. The composition according to claim 12, wherein the articular cartilage arthritis is osteoarthritis.

16. The composition according to claim 12, comprising the pro-chondrogenic agent, wherein the pro-chondrogenic agent comprises the NELL-1 protein or peptide.

17. The composition according to claim 15, which is in a formulation for injection.

18. The composition according to claim 12, which is in a formulation for transdermal delivery system.

19. The composition according to claim 12, wherein the subject is a human being.

20. A method of forming a composition, comprising:
providing an effective amount of an osteoinflammation preventive agent for cartilage arthritis, the osteoinflammation preventive agent comprising NELL-1 protein or peptide optionally in combination with a pro-chondrogenic agent, and
forming the composition.

21. The method according to claim 18, wherein the composition is according to any one of claims 13-19.


SEQUENCE LISTING (21 sequences)

SEQ ID NO: 1 - protein, 838 aa, Homo sapiens
MPMDLILVVWFCVCTARTAEDMKPPVLSLIWRTRVLKSLLQQSLQGVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 2 - protein, 816 aa, Homo sapiens
MESRVLLRTFCLIFGLGAVWGLGVDPSLQIDVLTELELGESTTGVRQVPGLHNGTKAFLFQDTPRSIKASTATAEQFFQKLRNKHEFTILVTLKQTHLNSGVILSIHHLDHRYLELESSGHRNEVRLHYRSGSHRPHTEVFPYILADDKWHKLSLAISASHLILHIDCNKIYERVVEKPSTDLPLGTTFWLGQRNNAHGYFKGIMQDVQLLVMPQGFIAQCPDLNRTCPTCNDFHGLVQKIMELQDILAKTSAKLSRAEQRMNRLDQCYCERTCTMKGTTYREFESWIDGCKNCTCLNGTIQCETLICPNPDCPLKSALAYVDGKCCKECKSICQFQGRTYFEGERNTVYSSSGVCVLYECKDQTMKLVESSGCPALDCPESHQITLSHSCCKVCKGYDFCSERHNCMENSICRNLNDRAVCSCRDGFRALREDNAYCEDIDECAEGRHYCRENTMCVNTPGSFMCICKTGYIRIDDYSCTEHDECITNQHNCDENALCFNTVGGHNCVCKPGYTGNGTTCKAFCKDGCRNGGACIAANVCACPQGFTGPSCETDIDECSDGFVQCDSRANCINLPGWYHCECRDGYHDNGMFSPSGESCEDIDECGTGRHSCANDTICFNLDGGYDCRCPHGKNCTGDCIHDGKVKHNGQIWVLENDRCSVCSCQNGFVMCRRMVCDCENPTVDLFCCPECDPRLSSQCLHQNGETLYNSGDTWVQNCQQCRCLQGEVDCWPLPCPDVECEFSILPENECCPRCVTDPCQADTIRNDITKTCLDEMNVVRFTGSSWIKHGTECTLCQCKNGHICCSVDPQCLQEL

SEQ ID NO: 3 - DNA, 15 bp, Artificial Sequence
  Description of Artificial Sequence Primer
gggtgtggtggggtg

SEQ ID NO: 4 - DNA, 15 bp, Artificial Sequence
  Description of Artificial Sequence Primer
tgctgtggtcacatc

SEQ ID NO: 5 - DNA, 15 bp, Artificial Sequence
  Description of Artificial Sequence Primer
tgctgtggtcacatc

SEQ ID NO: 6 - DNA, 15 bp, Artificial Sequence
  Description of Artificial Sequence Primer
gtctgtggtctgttt

SEQ ID NO: 7 - DNA, 15 bp, Artificial Sequence
  Description of Artificial Sequence Primer
gtctgtggtctgttt

SEQ ID NO: 8 - DNA, 19 bp, Artificial Sequence
  Description of Artificial Sequence Primer
ttgtccatggccacaacaa

SEQ ID NO: 9 - DNA, 19 bp, Artificial Sequence
  Description of Artificial Sequence Primer
atttgcatggtgatacatt

SEQ ID NO: 10 - DNA, 19 bp, Artificial Sequence
  Description of Artificial Sequence Primer
atttgcatggtgatacatt

SEQ ID NO: 11 - DNA, 19 bp, Artificial Sequence
  Description of Artificial Sequence Primer
atttgcatggtgatacatt

SEQ ID NO: 12 - DNA, 19 bp, Artificial Sequence
  Description of Artificial Sequence Primer
ctttccaggggagagaggg

SEQ ID NO: 13 - DNA, 19 bp, Artificial Sequence
  Description of Artificial Sequence Primer
agttccttggaagccaaga

SEQ ID NO: 14 - DNA, 19 bp, Artificial Sequence
  Description of Artificial Sequence Primer
agttccttggaagccaaga

SEQ ID NO: 15 - DNA, 19 bp, Artificial Sequence
  Description of Artificial Sequence Primer
tctcccctggaaaggacac

SEQ ID NO: 16 - DNA, 19 bp, Artificial Sequence
  Description of Artificial Sequence Primer
gcttccaaggaactctggg

SEQ ID NO: 17 - DNA, 19 bp, Artificial Sequence
  Description of Artificial Sequence Primer
gcttccaaggaactctggg

SEQ ID NO: 18 - DNA, 15 bp, Artificial Sequence
  Description of Artificial Sequence Primer
gattgtggttaagaa

SEQ ID NO: 19 - DNA, 19 bp, Artificial Sequence
  Description of Artificial Sequence Primer
tttcccgtggaccttccag

SEQ ID NO: 20 - DNA, 19 bp, Artificial Sequence
  Description of Artificial Sequence Primer
ttttccacggagcctctgc

SEQ ID NO: 21 - DNA, 19 bp, Artificial Sequence
  Description of Artificial Sequence Primer
ggctccgtggaaaactcac